(19) United States
(12) Patent Application Publication
(10) Pub. No.: US 2024/0402176 A1
(43) Pub. Date: Dec. 5, 2024

(54) DETECTION OF BLADDER CANCER IN MALES

(71) Applicant: RANDOX LABORATORIES LTD., Crumlin Antrim (GB)

(72) Inventors: Stephen Peter Fitzgerald, Crumlin Antrim (GB); Mark Ruddock, Crumlin Antrim (GB); John Lamont, Crumlin Antrim (GB)

(21) Appl. No.: 18/696,817

(22) PCT Filed: Sep. 29, 2022
(86) PCT No.: PCT/EP2022/077186

§ 371 Date: Mar. 28, 2024

(30) Foreign Application Priority Data
Oct. 1, 2021 (GB) 2114088.4

(51) Int. Cl. G01N 33/574; G01N 33/543

(52) U.S. Cl. CPC G01N 33/57407; G01N 33/54366; G01N 33/57488

(57) ABSTRACT

Haematuria is a considerable burden within primary and secondary care; too many haematuria patients are referred to secondary care for invasive and expensive investigations that could be managed in primary care. The current invention has identified biomarker combinations with utility for the diagnosis of bladder cancer in males. Using gender specific biomarker algorithms in combination with clinical risks that are associated with bladder cancer, would allow clinicians to better manage haematuria patients in primary care setting.

### INTRODUCTION

Bladder cancer is a leading cause of death worldwide. Bladder cancer is more than three times more common in men than women though the mortality rate in the latter is twice as great.

Cystoscopy and cytology are the gold standard used to diagnose bladder cancer. A cytological examination involves the examination of exfoliated cells in voided urine. This method has high specificity, and it is convenient to obtain a sample. However, cytology has poor sensitivity and is subjective at low cellular yield.

A cytological assessment is usually combined with flexibly cystoscopy. White light cystoscopy (WLC) allows direct observation of the bladder and biopsy of suspicious regions. However, recent publications have indicated that blue light cystoscopy (BLC) picked up 34% more tumours (e.g., carcinoma in situ (CIS)) than WLC. Furthermore, 20/53 patients (37.7%) with CIS lesions had negative cytology [1, 2]. Unfortunately, BLC has a higher false positive rate than WLC (39% vs. 31%, respectively) [1]. Cystoscopy has a sensitivity and specificity of 71% and 72%, respectively [3].

There are some disadvantages associated with cystoscopy, namely that it is expensive, causes patient discomfort, risk of infection and does not allow for upper urinary tract visualisation or for the detection of small areas of CIS. Increased number of bladder cancer recurrences are detected by cystoscopy when information on a positive urine test (cytology) is communicated to the urologist; but not when the cytology result is withheld [4].

No single biomarker or panel of biomarkers has yet achieved the levels of sensitivity and specificity required to reduce the frequency of cystoscopy needed for detection of bladder cancer. Over the last 10 years many bladder cancer biomarkers including Bladder Tumour Antigen (BTA), Nuclear Matrix Protein 22 (NMP22), telomerase and fibrinogen-degradation product(s) (FDP), have been evaluated against the gold standard cystoscopy with cytology however, the results demonstrate low specificity. Furthermore, the biomarkers are present in the urine of a large proportion of patients with underlying urological pathologies other than bladder cancer e.g., urinary infections (UTIs). NMP22 and BTA have FDA approval as point of care assays. However, NMP22 requires immediate stabilisation in urine, which is not always possible, and BTA can be confounded by blood present in the urine. New putative markers, such as survivin, hyaluronic acid, cytokeratin 8 and 18 and EGF, which have been shown to induce expression of the matrix metalloproteinase (MMP9) in some bladder cancer cells, have been proposed as bladder cancer biomarkers. However, none of the putative biomarkers have been bench-marked against the high specificity of urine cytology and the high sensitivity of the telomerase assay.

A lot of money and resources are used on giving low-risk patients cystoscopies who could be managed in primary care rather than increasing the wait for high-risk patients to get a cystoscopy. There is therefore a clinical need for a test that provides an accurate assessment which can allow a GP to rule out bladder cancer from the diagnosis without sending the patient for a cystoscopy.

### REFERENCES

[1] Fradet Y, et al., J Urol. 2007 July; 178(1): 68-73; discussion 73.
[2] Witjes J A, et al., Eur Urol. 2010 April; 57(4): 607-14.
[3] National Collaborating Centre for Cancer, Bladder Cancer: diagnosis and management; NICE Guidelines 2, February 2015, Page 78.
[4] Van der Aa M N, et al., J Urol. 2010 January; 183(1): 76-80.
[5] Bossuyt P M, Reitsma J B, Bruns D E, Gatsonis C A, Glasziou P P, Irwig L, et al., BMJ. 2015; 351.
[6] R Core Team. R: A Language and Environment for Statistical Computing 2018.

### BRIEF DESCRIPTION OF THE FIGURES

FIG. 1—Receiver Operating Curve for the biomarker combination with the highest AUC value for this dataset for the detection of Bladder Cancer in males: urine NSE, serum PAI-1/tPA, urine midkine, urine NGAL, urine MMP-9/TIMP-1 and serum prolactin: AUC 0.795 (sensitivity 71.8%, specificity 72.8%). Including urinary infection in the biomarker algorithm (upper line on graph) increased the AUC to 0.822 (sensitivity 77.9%, specificity 74.3%) (DeLong p=0.014).

### SUMMARY OF THE INVENTION

The present invention is based on the realisation that there are significant differences between the biomarkers required in the diagnosis of bladder cancer in males and females. The present invention therefore provides specific panels of biomarkers useful in the diagnosis of bladder cancer in male subjects.

In a first aspect of the invention there is a method for the detection of or for determining the risk of bladder cancer in a male patient comprising the steps of
(i) determining the level of a panel of biomarkers in a sample isolated from a male patient, said panel of biomarkers comprising one or both of prolactin and LIM and SH3 Protein 1 (LASP-1) and one or more additional biomarker selected from neuron specific enolase (NSE), Plasminogen Activator Inhibitor 1/Tissue Plasminogen Activator (PAI-1/tPA), Matrix Metalloprotein 9/Tissue Inhibitor of Metalloprotein 1 (MMP-9/TIMP-1), Neutrophil Gelatinase-Associated Lipocalin (NGAL), Midkine, Albumin: creatinine ratio (ACR), Bladder tumour antigen (BTA), Cluster of differentiation 44 (CD44), Carcinoembryonic antigen (CEA), Cytokeratin 18 (CK-18), Cytokeratin 20 (CK-20), Clusterin, Creatinine, C-reactive protein (CRP), C-X-C motif chemokine ligand 1 (CXCL1), C-X-C Motif Chemokine Ligand 16 (CXCL16), Cystatin B, Cystatin C, D-dimer, Epidermal growth factor (EGF), Fatty acid-binding protein A (FABP-A), FAS protein (FAS), Interferon gamma (IFNγ), Interleukin 1a (IL-1α), Interleukin 13 (IL-1B), Interleukin 2 receptor alpha chain (IL-2ra), Interleukin 6 (IL-6), Interleukin 7 (IL-7), Interleukin 8 (IL-8), Interleukin-10 (IL-10), Monocyte chemotactic protein 1 (MCP-1), Microalbumin, Matrix Metalloprotein-9/Neutrophil Gelatinase-Associated Lipocalin Complex (MMP-9/NGAL), Neuron specific enolase (NSE), Osmolality, Progranulin, Total urinary Protein (TUP), free prostate specific antigen to total prostate specific antigen ratio (PSA_TPSA), Protein S-100 A4 (S100A4), Thrombomodulin, Transforming growth factor beta 1 (TGFβ1), Tumour necrosis factor alpha (TNFα), Soluble tumour necrosis factor receptor 1 (sTNFR1), Soluble tumour necrosis factor receptor 2 (sTNFR2), Tissue Type Plasminogen Activator (TPA), Vascular Endothelial Growth Factor (VEGF) and Cholesterol;
(ii) assessing the presence or risk of bladder cancer in the male patient wherein detection of an altered level of the biomarkers compared to a normal control indicates the presence or the risk of cancer in the male patient from whom the sample has previously been isolated.

Preferred combinations of the current invention include prolactin and NSE and at least one additional biomarker selected from PAI-1/tPA, NGAL, MMP-9/TIMP-1, and midkine.

In a second aspect of the invention there is a solid support material comprising binding molecules attached thereto, said binding molecules having affinity specific for prolactin and optionally, separately, LASP-1 and one or more additional biomarkers of NSE, PAI-1/tPA, Midkine, NGAL, and MMP-9/TIMP-1, with the binding molecules for each being in discrete locations on the support material.

In a third aspect of the invention there is a method for the detection of or determining the risk of bladder cancer in a male patient comprising the steps of
(i) determining that a male patient does not have an infection;
(ii) detecting the presence of Prolactin and one or more additional biomarkers in a sample isolated from the male patient, wherein said one or more biomarkers are selected from LASP-1, NSE, PAI-1/tPA, Midkine, NGAL, CXCL16 and MMP-9/TIMP-1;
(iii) assessing the presence or risk of bladder cancer in the male patient wherein detection of an elevated presence of the biomarkers compared to a normal control indicates the presence or the risk of cancer in the male patient from whom the sample has previously been isolated.

### Materials and Methods

### HABIO Patients

Patients (N=675) presenting with haematuria and undergoing cystoscopy were recruited to the HABIO study between 2013 and 2016 at Northern Ireland hospitals-Ulster, Craigavon, and Belfast City.

Research nurses (RNs) collected and recorded demographic, clinicopathological data and information about treatments on a Recruitment Form. The patient was asked about their lifestyle, hobbies and pastimes, the number of times they pass urine and whether they have dysuria, their occupation(s), current medication(s) and whether they have ever been exposed to hazardous chemicals. Weight, height, and blood pressure were recorded. RNs collected and recorded investigation results for the Final Review Form.

All BC cases (N=201)>40 and ≤80 years were recruited with pathologically proven (newly diagnosed (n=146, 72.6%) or recurrent (n=55, 27.4%)) BC at pre-assessment clinics, from inpatients or at planned cystoscopy clinics. Final diagnosis of subjects was determined as follows. A consultant pathologist undertook a review of diagnostic pathology for all patients. Pathological grade and stage and presence/absence of inflammatory infiltrate together with other relevant information were recorded. A consultant cytopathologist undertook a review of diagnostic cytology and recorded diagnosis and noted the presence/absence of inflammatory cells. Cytology was assessed on Papanicolaou and Giemsa-stained preparations. Urinary infection was diagnosed based on patient clinical history, biomarkers, and dipstick analysis.

Control patients (N=474; infection (n=221); BPE (n=213); healthy (n=30); no diagnosis (n=119); other cancers (n=3); other benign conditions (n=24); prostate cancer (n=10)) were recruited from haematuria clinics following negative cystoscopy and negative findings from BC investigations. Written informed consent was obtained from all patients and samples were collected in the outpatient setting. HABIO inclusion and exclusion criteria are described below:

### Inclusion Criteria

### Bladder Cancer Patients.

Written informed consent to participate in the study
Aged between 40 and 80 years.
Current haematuria or a history of haematuria
Cystoscopy within the last 6 months or planned cystoscopy
No chemo- or radio-therapy in the three weeks prior to recruitment.
No previous history of cancers other than bladder cancer.
Suspicion of bladder cancer or proven bladder cancer

### Control Patients

Written informed consent to participate in the study
No previous history of cancer
Of the same gender, approximate age, and smoking status (where possible) to a bladder cancer patient already recruited to HABIO.
Current haematuria or a history of haematuria
Negative cystoscopy within the last 3 months, but at least 48h after the procedure
No chemo- or radio-therapy in the three weeks prior to recruitment

### Exclusion Criteria

Bladder cancer patients:
No written informed consent to participate in the study
Aged <40 or >85 years
No history of haematuria
No recent or planned cystoscopy.
Chemo- or radio-therapy in the three weeks prior to recruitment.
Previous history of cancer(s), other than bladder cancer.
No suspicion of bladder cancer or proven bladder cancer

### Control Patients

No written informed consent to participate in the study
Previous history of any cancer
Not of the same gender, approximate age and smoking status of a patients already recruited as a bladder cancer patient.
No history of haematuria
No recent or planned cystoscopy.
Chemo- or radio-therapy in the three weeks prior to recruitment.

The study was approved by the Office for Research Ethics Committees Northern Ireland (ORECNI 11/NI/0164), reviewed by hospital review boards, registered with Cancer Research UK (ISRCTN25823942) and conducted according to Standards for Reporting of Diagnostic Accuracy (STARD) [5].

### HABIO Patient Samples

Patient urine (25 ml) and blood (35 ml) samples were collected and processed by a RN/technician. Whole blood was allowed to clot, and the resulting serum was removed, aliquoted and frozen. Unfiltered and uncentrifuged urine samples were immediately aliquoted and frozen at −80° C. Frozen samples were transported on dry ice to Randox Laboratories Ltd, Crumlin, UK. Urine and serum samples were thawed on ice and centrifuged (1200×g, 10 minutes, 4° C.) to remove particulate matter prior to analysis.

### Biomarker Measurement

All patient samples were run in triplicate and the results are expressed as mean±SD (n=3). Biochip Array Technology (Randox Laboratories Ltd., Crumlin, Northern Ireland, UK) was used for the simultaneous detection of multiple analytes from a single patient sample (urine). The technology is based on the Randox Biochip, a 9 mm² solid substrate supporting an array of discrete test regions with immobilized, antigen-specific antibodies. Following antibody activation with assay buffer, standards and samples were added and incubated at 37° C. for 60 minutes, then placed in a thermo-shaker at 370 rpm for 60 minutes. Antibody conjugates (HRP) were added and incubated in the thermo-shaker at 370 rpm for 60 minutes. The chemiluminescent signals formed after the addition of luminol (1:1 ratio with conjugate) were detected and measured using digital imaging technology and compared with that from a calibration curve to calculate concentration of the analytes in the samples. The analytical sensitivity of the biochip(s) was as follows: cystatin C 0.60 ng/ml; EGF 2.5 pg/ml; IFNγ 2.1 pg/ml; IL-2 4.8 pg/ml; IL-2Ra 0.12 ng/ml; IL-23 13.0 pg/ml; IL-3 8.78 pg/ml; IL-4 6.6 pg/ml; IL-6 1.2 pg/ml; IL-6R 0.62 ng/ml; IL-7 1.11 pg/ml; IL-8 7.9 pg/ml; IL-10 1.1 pg/ml; IL-12p70 2.61 pg/ml; IL-13 5.23 pg/ml; VEGF 14.6 pg/ml; TNFα 4.4 pg/ml; IL-1α 0.8 pg/ml; IL-1β 1.6 pg/ml; MCP-1 13.2 pg/ml; NSE 0.26 ng/ml; NGAL 17.8 ng/ml; sTNFR1 0.24 ng/ml; D-dimer 2.1 ng/ml; sTNFR2 0.2 ng/ml; and CRP 0.67 mg/ml. Functional sensitivity for CEA and PSA (free and total) on the biochip were 0.29, 0.02 and 0.45 ng/ml, respectively. All biochips were run on an Evidence Investigator analyser according to manufacturer's instructions (Randox Laboratories Ltd, Crumlin, UK). The analytical sensitivity for HDL, LDL and cholesterol were as follows: direct HDL cholesterol (HDL) 0.189 mmol/l (7.30 mg/dl), direct LDL cholesterol (LDL) 0.189 mmol/l (7.30 mg/dl) and cholesterol 0.865 mmol/l (33.4 mg/dl), respectively. HDL, LDL, and cholesterol were run on a Daytona analyser (Randox Laboratories Ltd, Crumlin, UK). The analytical sensitivity for urinary microalbumin was 5.11 mg/l. Microalbumin was analysed on a Daytona Plus analyser (Randox, Crumlin, UK). The analytical sensitivity for prolactin was 6.52 mIU/I. Prolactin was run on an Evidence Evolution analyser (Randox, Crumlin, UK). The analytical sensitivity for cystatin C was 0.4 mg/l. Serum Cystatin C was run on a Daytona analyser (RCLS, Antrim, UK). Triglycerides were run on a Daytona analyser (RCLS, Antrim, UK). Creatinine (μmol/l) measurements were performed by Randox Testing Services, Crumlin, UK, using a quantitative in vitro diagnostic assay from Randox (Crumlin, UK) on a Daytona analyser, according to manufacturers' instructions (Randox).

### Commercial ELISA Kits

The following biomarkers were detected using commercially available ELISA kits, as per manufactures instructions; all patient samples were run in triplicate: 8-hydroxy 2 deoxyguanosine (8OHdG), minimum detectable difference (MDD) 0.1 ng/ml (Cell Biolabs, San Diego, US); Bladder tumour antigen (BTA), MDD 0.65 U/ml (Polymedco, New York, US); Cluster of differentiation 44 (CD44), MDD<0.113 ng/ml (Abcam, Cambridge, UK); UBC II (CK-8, CK-18), MDD 0.1 ng/ml (IDL, Bromma, Sweden); Cytokeratin-20, MDD 0.1 ng/ml (CK-20) (BlueGene, Shanghai, China); Clusterin, MDD 0.189 ng/ml (R&D Systems, Abingdon, UK); CXCL16, MDD 0.007 ng/ml (R&D Systems, Abingdon, UK); Cystatin B, MDD 0.013 ng/ml (R&D Systems, Abingdon, UK); Epithelial growth factor (EGF), MDD 25 pg/ml (Randox, Antrim, UK); Fatty acid-binding protein-adipose (FABP-A), MDD 0.05 ng/ml (Biovendor, Abingdon, UK); Tumour necrosis factor receptor superfamily member 6 (FAS), 5 μg/ml (RayBio, Georgia, US); Hyaluronic acid (HAD), MDD 0.1 U/I (MyBioSource, San Diego, US); C-X-C ligand 1 motif/growth regulated alpha protein (CXCL1/GROα) MDD 10 μg/ml (R&D Systems, Abingdon, UK); Interleukin 18 (IL-18), MDD 42.8 pg/ml (Randox, Antrim, UK); LIM and SH3 domain (LASP-1), MDD 6.25 pg/ml (Cusabio, Houston, US); Muscle type-2 pyruvate kinase (M2-PK), MDD 3.4 ng/ml (Randox, Antrim, UK); Caspase-cleaved CK-18 fragments (M30), MDD 20 U/L (Previva, Paudex, Switzerland); Midkine, MDD 8 μg/ml (CellMid, Sydney, Australia); Matrix metallopeptidase 9/Neutrophil gelatinase-associated lipocalin complex (MMP-9/NGAL), MDD 0.013 ng/ml (R&D Systems, Abingdon, UK); Matrix metallopeptidase 9/Tissue inhibitor of metallopeptidase-1 complex (MMP-9/TIMP-1), MDD 0.0469 ng/ml (R&D Systems, Abingdon, UK); Plasminogen activator inhibitor-1/Tissue plasminogen activator complex (PAI-1/tPA), MDD 0.04 ngml (AssayPro, Missouri, US); Phospho-extracellular signal-related kinase (PERK), MDD 18.75 pg/ml (MyBioSource, San Diego, UK); Progranulin, MDD 0.17 ng/ml (R&D Systems, Abingdon, UK), S100 calcium-binding protein A4 (S100A4), MDD 0.225 ng/ml (Cusabio, Houston, US); Transforming growth factor beta-1 (TGFβ1), MDD 4.61 pg/ml (R&D Systems, Abingdon, UK); Thrombomodulin, MDD 7.82 pg/ml (R&D Systems, Abingdon, UK) and Tissue plasminogen activator (TPA), MDD 0.01 ng/ml (Abcam, Cambridge, UK).

### Point of Care Assays and Investigations

At recruitment, patient urine samples were collected prior to cytoscopic examination and evaluated using the point of care test (POCT) Nuclear Matrix Protein 22 (NMP22) (BladderChek, Alere, US), according to manufacturer's instructions (MDD<10 U/ml were negative). Aution sticks 10EA used for dipstick urinalysis were interpreted using the PocketChem analyser (Arkray Inc, Japan).

### Osmolality

Osmolality (mOsm) was determined using a L□ser Micro-osmometer according to manufacturer's instructions (Löser Messtechnik, Berlin, Germany).

### Total Urinary Protein (Bradford Assay)

Total urinary protein levels (mg/ml) were determined, in triplicate, by Bradford assay (Pierce, Rockford, IL, USA) using a stock solution of BSA (Sigma) as standard (1 mg/ml). Patient urine samples (10 μl/patient), after centrifugation (1200 g, 10 minutes, 4° C.), were mixed with Bradford reagent (1 ml) and allowed to stand for 5 minutes. The samples were read on a Hitachi Spectrophotometer (Model No. U-2800) at A595 nm. Total urinary protein was determined using a BSA calibration chart.

### Statistical Analysis

Statistical analyses were undertaken using IBM SPSSv25 and R [6]. Continuous clinical characteristics and biomarker data were analysed using Wilcoxon mean rank sum and descriptive characteristics were analysed using Chi-Squared contingency test to identify which factors were differentially expressed between control and BC. Statistical significance was taken at the p<0.05 level and results are presented as mean±SD where appropriate (Tables 1-3). Biomarkers differentially expressed between groups were investigated by forward and backward Wald binary logistic regression and least absolute shrinkage and selection operator (Lasso) to identify algorithms to diagnose BC. Comparisons between AUROC were undertaken using the DeLong test.

### Results

BC patients were older and were more likely to present with macroscopic haematuria. The male to female ratio for haematuria was 2.6:1.0 and for BC was 3.0:1.0. HABIO patients with BC smoked more and had higher total tar exposure. Loss of bladder control was noted for both males and females however, this was only significant for females. Alcohol consumption was not significantly different between groups.

### Medications

Statins and PPIs were the most common medication classes. There was no difference in the mean number of medications taken by control or BC patients (5.2±3.8 vs. 4.9±3.7, p=0.462, respectively).

### Point of Care Assays and Investigations

NMP22 was positive for G2 (n=11/109 (10%)) and G3 (n=28/81 (35%)) BCs and failed to detect any G1 tumours (n=0/6). Three urinary markers were identified by aution dipstick as significantly different between control and BC: namely, protein (p<0.001), specific gravity (p=0.029) and blood (p<0.001).

### HABIO Biomarker Measurements

Urine and serum biomarker results are described in Tables 1-3. It was noted that some biomarkers were gender specific. Thus, separate gender-specific biomarker algorithms were identified.

### Male BC Biomarker Algorithm

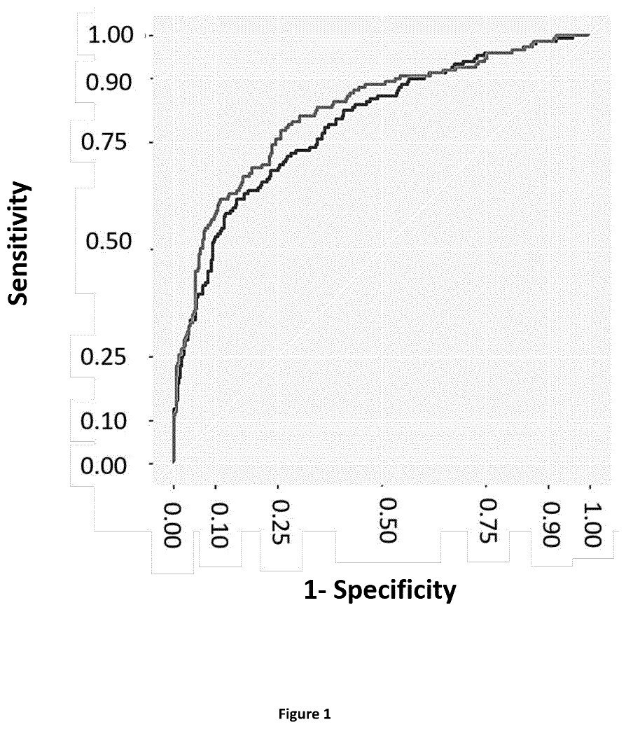

The following biomarker combination was identified as the combination with the highest AUC value for this dataset for the detection of BC in males: urine NSE, serum PAI-1/tPA, urine midkine, urine NGAL, urine MMP-9/TIMP-1 and serum prolactin: AUC 0.795 (sensitivity 71.8%, specificity 72.8%). Including urinary infection in the biomarker algorithm increased the AUC to 0.822 (sensitivity 77.9%, specificity 74.3%) (DeLong p=0.014) (FIG. 1).

TABLE 1
Significance of individual biomarkers between controls and
bladder cancer patients when not separated by gender

| | ALL | | |
|---|---|---|---|
| Biomarker | Con | BC | P |
| (matrix) + units | (Status at FD) | (Status at FD) | value |
| 80HdG (urine) | 57.3 ± 40.4 | 62.7 ± 40.9 | 0.058 |
| ng/ml | (n = 473) | (n = 194) | |
| ACR (urine) | 4.9 ± 10.5 | 7.7 ± 10.1 | <0.001 |
| | (n = 473) | (n = 199) | |
| BTA (urine) | 17.5 ± 30.2 | 38.2 ± 42.0 | <0.001 |
| U/ml | (n = 473) | (n = 199) | |
| CD44 (serum) | 136.8 ± 58.9 | 155.6 ± 75.5 | 0.019 |
| ng/ml | (n = 472) | (n = 199) | |
| CEA (serum) | 2.1 ± 1.9 | 3.3 ± 11.8 | 0.011 |
| ng/ml | (n = 473) | (n = 199) | |
| CK-18 (urine) | 28.0 ± 84.9 | 158.4 ± 463.8 | <0.001 |
| ng/ml | (n = 460) | (n = 176) | |
| CK-20 (urine) | 0.198 ± 0.130 | 0.188 ± 0.183 | 0.004 |
| ng/ml | (n = 473) | (n = 194) | |
| Clusterin (urine) | 243.4 ± 324.7 | 503.4 ± 564.7 | <0.001 |
| ng/ml | (n = 473) | (n = 199) | |
| Creatinine (urine) | 91.8 ± 57.3 | 114.1 ± 71.1 | <0.001 |
| μmol/l | (n = 473) | (n = 199) | |
| CRP (urine) | 1.4 ± 0.5 | 1.5 ± 0.7 | 0.157 |
| mg/ml | (n = 473) | (n = 199) | |
| CRP (serum) | 3.28 ± 8.11 | 3.91 ± 7.56 | 0.001 |
| mg/ml | (n = 458) | (n = 190) | |
| CXCL16 (urine) | 0.06 ± 0.32 | 0.13 ± 0.27 | <0.001 |
| ng/ml | (n = 472) | (n = 193) | |
| Cystatin B (urine) | 8.9 ± 18.2 | 20.0 ± 29.3 | <0.001 |
| ng/ml | (n = 473) | (n = 199) | |
| Cystatin C (urine) | 29.8 ± 43.5 | 40.9 ± 40.3 | <0.001 |
| ng/ml | (n = 473) | (n = 197) | |
| Cystatin C | 0.958 ± 0.338 | 0.974 ± 0.281 | 0.307 |
| (serum) ng/ml | (n = 470) | (n = 197) | |
| D-dimer (urine) | 49.3 ± 206.9 | 215.4 ± 487.5 | <0.001 |
| ng/ml | (n = 473) | (n = 199) | |
| EGF (urine) | 8833.8 ± 11605.4 | 15673.4 ± 19300.7 | <0.001 |
| pg/ml | (n = 473) | (n = 197) | |
| EGF (serum) | 16.3 ± 17.5 | 26.1 ± 27.0 | <0.001 |
| pg/ml | (n = 469) | (n = 197) | |
| FABP-A (serum) | 35.0 ± 23.9 | 39.3 ± 31.7 | 0.282 |
| ng/ml | (n = 473) | (n = 199) | |
| FAS (urine) | 25.1 ± 77.6 | 53.4 ± 104.4 | <0.001 |
| pg/ml | (n = 473) | (n = 199) | |
| GRO (CXCL1) | 95.6 ± 101.5 | 101.7 ± 90.8 | 0.001 |
| (serum) pg/ml | (n = 473) | (n = 199) | |
| HAD (serum) | 0.260 ± 0.480 | 0.253 ± 0.712 | 0.08 |
| U/l | (n = 473) | (n = 197) | |
| IFNγ(urine) | 2.028 ± 1.096 | 2.036 ± 0.685 | 0.001 |
| pg/ml | (n = 473) | (n = 199) | |
| IFNγ (serum) | 2.202 ± 2.626 | 2.005 ± 0.799 | 0.753 |
| pg/ml | (n = 469) | (n = 197) | |
| IL-1α (urine) | 4.5 ± 17.9 | 23.6 ± 69.9 | <0.001 |
| pg/ml | (n = 473) | (n = 199) | |
| IL-1α(serum) | 0.901 ± 1.096 | 0.853 ± 0.227 | 0.157 |
| pg/ml | (n = 469) | (n = 197) | |
| IL-1β (urine) | 14.6 ± 44.3 | 26.3 ± 62.1 | 0.001 |
| pg/ml | (n = 473) | (n = 199) | |
| IL-1β (serum) | 1.4 ± 1.2 | 1.2 ± 0.2 | 0.708 |
| pg/ml | (n = 469) | (n = 197) | |
| IL-2 (urine) | 4.7 ± 2.2 | 4.9 ± 3.0 | 0.562 |
| pg/ml | (n = 473) | (n = 199) | |
| IL-2 (serum) | 5.2 ± 4.7 | 4.5 ± 1.0 | 0.075 |
| pg/ml | (n = 469) | (n = 197) | |
| IL-3 (urine) | 8.2 ± 4.2 | 8.4 ± 5.1 | 0.328 |
| pg/ml | (n = 472) | (n = 199) | |
| IL-4 (urine) | 3.553 ± 1.589 | 3.505 ± 1.141 | 0.225 |
| pg/ml | (n = 473) | (n = 199) | |
| IL-4 (serum) | 3.609 ± 2.433 | 3.309 ± 0.732 | 0.073 |
| pg/ml | (n = 469) | (n = 197) | |
| IL-6 (urine) | 21.4 ± 96.1 | 74.5 ± 186.8 | <0.001 |
| pg/ml | (n = 473) | (n = 199) | |
| IL-6 (serum) | 4.06 ± 7.59 | 4.23 ± 4.77 | 0.007 |
| pg/ml | (n = 469) | (n = 197) | |
| IL-7 (urine) | 3.5 ± 3.3 | 4.6 ± 3.1 | <0.001 |
| pg/ml | (n = 472) | (n = 199) | |
| IL-8 (urine) | 245.1 ± 464.3 | 425.7 ± 556.5 | <0.001 |
| pg/ml | (n = 473) | (n = 199) | |
| IL-8 (serum) | 14.0 ± 12.3 | 14.7 ± 10.8 | 0.388 |
| pg/ml | (n = 469) | (n = 197) | |
| IL-10 (urine) | 1.101 ± 0.612 | 1.146 ± 1.043 | 0.386 |
| pg/ml | (n = 473) | (n = 199) | |
| IL-10 (serum) | 2.35 ± 21.48 | 1.34 ± 1.75 | 0.251 |
| pg/ml | (n = 469) | (n = 197) | |
| IL-12p70 (urine) | 3.405 ± 2.867 | 3.591 ± 5.122 | 0.018 |
| pg/ml | (n = 472) | (n = 199) | |
| IL-13 (urine) | 6.4 ± 6.1 | 8.1 ± 14.1 | 0.019 |
| pg/ml | (n = 472) | (n = 199) | |
| IL-18 (serum) | 225.5 ± 434.3 | 159.3 ± 291.3 | 0.925 |
| pg/ml | (n = 471) | (n = 198) | |
| IL-23 (urine) | 0.013 ± 0.021 | 0.013 ± 0.007 | 0.48 |
| pg/ml | (n = 472) | (n = 199) | |
| LASP-1 (serum) | 394.5 ± 300.8 | 501.1 ± 552.9 | 0.026 |
| pg/ml | (n = 473) | (n = 199) | |
| M30 (serum) | 175.2 ± 139.7 | 170.6 ± 137.2 | 0.984 |
| U/l | (n = 473) | (n = 199) | |
| M2PK (serum) | 32.9 ± 59.3 | 31.6 ± 16.9 | 0.83 |
| ng/ml | (n = 472) | (n = 198) | |
| MCP-1 (urine) | 135.5 ± 185.2 | 207.5 ± 235.1 | <0.001 |
| pg/ml | (n = 473) | (n = 199) | |
| MCP-1 (serum) | 387.7 ± 131.3 | 400.8 ± 148.6 | 0.308 |
| pg/ml | (n = 469) | (n = 197) | |
| Microalbumin | 33.6 ± 59.4 | 69.1 ± 83.8 | <0.001 |
| (urine) mg/l | (n = 473) | (n = 199) | |
| Midkine (urine) | 327.4 ± 521.5 | 888.8 ± 1232.4 | <0.001 |
| pg/ml | (n = 473) | (n = 199) | |
| MMP-9 (urine) | 15.8 ± 39.6 | 22.3 ± 53.9 | 0.64 |
| ng/ml | (n = 473) | (n = 199) | |
| MMP-9/NGAL | 4.6 ± 10.6 | 7.3 ± 12.4 | <0.001 |
| (urine) ng/ml | (n = 473) | (n = 199) | |
| MMP-9/TIMP-1 | 0.6 ± 3.4 | 2.2 ± 6.6 | <0.001 |
| (urine) ng/ml | (n = 473) | (n = 199) | |
| NGAL (urine) | 408.3 ± 501.8 | 515.8 ± 593.0 | 0.021 |
| ng/ml | (n = 473) | (n = 199) | |
| NSE (urine) | 0.6 ± 1.8 | 6.5 ± 21.4 | <0.001 |
| ng/ml | (n = 473) | (n = 199) | |
| Osmolality (urine) | 530.4 ± 231.6 | 580.8 ± 188.7 | 0.007 |
| mOsm/l | (n = 473) | (n = 199) | |
| PAI-1/tPA (serum) | 8.2 ± 7.0 | 12.6 ± 9.8 | <0.001 |
| ng/ml | (n = 473) | (n = 199) | |
| PERK (urine) | 393.2 ± 335.2 | 412.2 ± 367.4 | 0.589 |
| pg/ml | (n = 472) | (n = 194) | |
| Progranulin (urine) | 13.3 ± 10.3 | 18.2 ± 18.3 | <0.001 |
| ng/ml | (n = 473) | (n = 196) | |
| Prolactin (serum) | 260.9 ± 248.0 | 227.9 ± 162.0 | 0.001 |
| mIU/l | (n = 468) | (n = 196) | |
| Protein (urine) | 0.1 ± 0.3 | 0.4 ± 0.7 | <0.001 |
| mg/ml | (n = 473) | (n = 199) | |
| PSA/TPSA (serum) | 2.4 ± 4.1 | 2.3 ± 4.5 | 0.533 |
| ng/ml | (n = 473) | (n = 199) | |
| S100A4 (serum) | 54.3 ± 34.8 | 61.7 ± 45.0 | 0.145 |
| ng/ml | (n = 473) | (n = 199) | |
| sIL-2Ra (serum) | 0.121 ± 0.098 | 0.124 ± 0.061 | 0.037 |
| ng/ml | (n = 473) | (n = 199) | |
| sIL-6R (urine) | 0.564 ± 0.062 | 0.562 ± 0.024 | 0.489 |
| ng/ml | (n = 473) | (n = 199) | |
| TGFβ1 (urine) | 39.3 ± 128.0 | 118.0 ± 354.8 | <0.001 |
| pg/ml | (n = 473) | (n = 199) | |
| Thrombomodulin | 15.1 ± 10.1 | 18.2 ± 10.3 | <0.001 |
| (urine) pg/ml | (n = 473) | (n = 199) | |
| TNFα (urine) | 4.3 ± 2.3 | 5.7 ± 4.8 | <0.001 |
| pg/ml | (n = 473) | (n = 199) | |
| TNFα (serum) | 4.3 ± 2.8 | 4.0 ± 0.9 | 0.818 |
| pg/ml | (n = 469) | (n = 197) | |
| sTNFRI (urine) | 0.8 ± 0.8 | 1.0 ± 0.8 | <0.001 |
| ng/ml | (n = 473) | (n = 199) | |
| sTNRFII (urine) | 1.7 ± 2.1 | 1.9 ± 2.0 | 0.02 |
| ng/ml | (n = 473) | (n = 199) | |
| TPA (urine) | 1.6 ± 3.6 | 2.9 ± 4.6 | <0.001 |
| ng/ml | (n = 473) | (n = 197) | |
| VEGF (urine) | 147.6 ± 209.2 | 335.0 ± 526.2 | <0.001 |
| pg/ml | (n = 473) | (n = 199) | |
| VEGF (serum) | 242.5 ± 161.9 | 293.1 ± 202.9 | 0.002 |
| pg/ml | (n = 469) | (n = 197) | |
| HDL (serum) | 1.2 ± 0.4 | 1.2 ± 0.4 | 0.242 |
| mmol/l | (n = 459) | (n = 191) | |
| LDL (serum) | 2.8 ± 1.0 | 2.6 ± 1.1 | 0.018 |
| mmol/l | (n = 459) | (n = 191) | |
| Triglycerides (serum) | 1.7 ± 1.0 | 1.4 ± 0.7 | 0.003 |
| mmol/l | (n = 459) | (n = 191) | |
| Cholesterol (serum) | 4.8 ± 1.2 | 4.5 ± 1.2 | 0.013 |
| mmol/l | (n = 459) | (n = 191) | |

TABLE 2
Significance of individual biomarkers between
male controls and male bladder cancer patients

| | Male | | |
|---|---|---|---|
| Biomarker | Con | BC | p |
| (matrix) + units | (Status at FD) | (Status at FD) | value |
| 80HdG (urine) | 62.7 ± 41.4 | 63.8 ± 40.3 | 0.701 |
| ng/ml | (n = 334) | (n = 147) | |
| ACR (urine) | 5.7 ± 11.9 | 7.8 ± 10.5 | 0.001 |
| | (n = 334) | (n = 150) | |
| BTA (urine) | 21.0 ± 33.2 | 38.3 ± 42.8 | <0.001 |
| U/ml | (n = 334) | (n = 150) | |
| CD44 (serum) | 131.5 ± 54.9 | 149.5 ± 71.9 | 0.057 |
| ng/ml | (n = 333) | (n = 151) | |
| CEA (serum) | 2.1 ± 2.0 | 3.6 ± 13.5 | 0.086 |
| ng/ml | (n = 334) | (n = 151) | |
| CK-18 (urine) | 33.5 ± 97.9 | 178.8 ± 513.7 | <0.001 |
| ng/ml | (n = 323) | (n = 131) | |
| CK-20 (urine) | 0.2 ± 0.1 | 0.2 ± 0.2 | 0.008 |
| ng/ml | (n = 334) | (n = 147) | |
| Clusterin (urine) | 282.8 ± 357.9 | 524.0 ± 589.4 | <0.001 |
| ng/ml | (n = 334) | (n = 150) | |
| Creatinine (urine) | 103.4 ± 58.5 | 120.1 ± 72.2 | 0.014 |
| μmol/l | (n = 334) | (n = 150) | |
| CRP (urine) | 1.4 ± 0.6 | 1.5 ± 0.6 | 0.388 |
| mg/ml | (n = 334) | (n = 150) | |
| CRP (serum) | 3.3 ± 8.8 | 4.1 ± 8.1 | 0.001 |
| mg/ml | (n = 325) | (n = 144) | |
| CXCL16 (urine) | 0.1 ± 0.4 | 0.1 ± 0.3 | <0.001 |
| ng/ml | (n = 334) | (n = 146) | |
| Cystatin B (urine) | 6.7 ± 16.3 | 18.0 ± 29.1 | <0.001 |
| ng/ml | (n = 334) | (n = 150) | |
| Cystatin C (urine) | 34.2 ± 48.5 | 41.5 ± 39.8 | 0.002 |
| ng/ml | (n = 334) | (n = 149) | |
| Cystatin C (serum) | 1.0 ± 0.3 | 1.0 ± 0.3 | 0.326 |
| ng/ml | (n = 332) | (n = 150) | |
| D-dimer (urine) | 64.3 ± 240.8 | 231.1 ± 509.1 | <0.001 |
| ng/ml | (n = 334) | (n = 150) | |
| EGF (urine) | 9495.2 ± 11564.4 | 15160.8 ± 18669.4 | 0.005 |
| pg/ml | (n = 334) | (n = 148) | |
| EGF (serum) | 15.6 ± 16.6 | 23.5 ± 23.8 | <0.001 |
| pg/ml | (n = 331) | (n = 150) | |
| FABP-A (serum) | 30.0 ± 20.6 | 35.3 ± 29.0 | 0.107 |
| ng/ml | (n = 334) | (n = 151) | |
| FAS (urine) | 29.2 ± 91.6 | 55.9 ± 101.3 | <0.001 |
| pg/ml | (n = 334) | (n = 150) | |
| GRO (CXCL1) | 89.3 ± 98.6 | 93.2 ± 70.2 | 0.001 |
| (serum) pg/ml | (n = 334) | (n = 151) | |
| HAD (serum) | 0.3 ± 0.6 | 0.2 ± 0.2 | 0.963 |
| U/l | (n = 334) | (n = 149) | |
| IFNγ (urine) | 2.0 ± 1.2 | 2.0 ± 0.4 | 0.005 |
| pg/ml | (n = 334) | (n = 150) | |
| IFNγ (serum) | 2.2 ± 2.6 | 1.9 ± 0.6 | 0.334 |
| pg/ml | (n = 331) | (n = 150) | |
| IL-1α (urine) | 3.8 ± 19.5 | 20.5 ± 65.5 | <0.001 |
| pg/ml | (n = 334) | (n = 150) | |
| IL-1α (serum) | 0.9 ± 0.3 | 0.8 ± 0.2 | 0.724 |
| pg/ml | (n = 331) | (n = 150) | |
| IL-1β (urine) | 17.3 ± 49.7 | 24.1 ± 60.3 | 0.178 |
| pg/ml | (n = 334) | (n = 150) | |
| IL-1β (serum) | 1.4 ± 1.2 | 1.2 ± 0.2 | 0.399 |
| pg/ml | (n = 331) | (n = 150) | |
| IL-2 (urine) | 4.7 ± 2.6 | 4.8 ± 2.9 | 0.451 |
| pg/ml | (n = 334) | (n = 150) | |
| IL-2 (serum) | 5.2 ± 5.1 | 4.5 ± 0.9 | 0.054 |
| pg/ml | (n = 331) | (n = 150) | |
| IL-3 (urine) | 8.3 ± 5.0 | 8.5 ± 5.8 | 0.459 |
| pg/ml | (n = 334) | (n = 150) | |
| IL-4 (urine) | 3.6 ± 1.8 | 3.4 ± 0.8 | 0.979 |
| pg/ml | (n = 334) | (n = 150) | |
| IL-4 (serum) | 3.5 ± 1.5 | 3.3 ± 0.8 | 0.072 |
| pg/ml | (n = 331) | (n = 150) | |
| IL-6 (urine) | 27.5 ± 113.3 | 75.3 ± 190.0 | 0.003 |
| pg/ml | (n = 334) | (n = 150) | |
| IL-6 (serum) | 4.6 ± 8.8 | 4.5 ± 5.1 | 0.052 |
| pg/ml | (n = 331) | (n = 150) | |
| IL-7 (urine) | 4.0 ± 3.6 | 4.8 ± 3.0 | 0.001 |
| pg/ml | (n = 334) | (n = 150) | |
| IL-8 (urine) | 260.2 ± 497.5 | 403.5 ± 567.1 | <0.001 |
| pg/ml | (n = 334) | (n = 150) | |
| IL-8 (serum) | 14.3 ± 13.7 | 14.5 ± 11.3 | 0.835 |
| pg/ml | (n = 331) | (n = 150) | |
| IL-10 (urine) | 1.1 ± 0.7 | 1.1 ± 0.3 | 0.983 |
| pg/ml | (n = 334) | (n = 150) | |
| IL-10 (serum) | 2.8 ± 25.6 | 1.2 ± 0.6 | 0.023 |
| pg/ml | (n = 331) | (n = 150) | |
| IL-12p70 (urine) | 3.5 ± 3.3 | 3.9 ± 5.9 | 0.317 |
| pg/ml | (n = 334) | (n = 150) | |
| IL-13 (urine) | 6.7 ± 7.1 | 8.4 ± 16.2 | 0.901 |
| pg/ml | (n = 334) | (n = 150) | |
| IL-18 (serum) | 223.5 ± 425.6 | 180.1 ± 322.6 | 0.419 |
| pg/ml | (n = 333) | (n = 150) | |
| IL-23 (urine) | 0.0 ± 0.0 | 0.0 ± 0.0 | 0.532 |
| pg/ml | (n = 334) | (n = 150) | |
| LASP-1 (serum) | 388.8 ± 298.4 | 531.0 ± 609.6 | 0.012 |
| pg/ml | (n = 334) | (n = 151) | |
| M30 (serum) | 178.2 ± 133.7 | 178.8 ± 149.3 | 0.907 |
| U/l | (n = 334) | (n = 151) | |
| M2PK (serum) | 30.7 ± 14.8 | 31.8 ± 16.8 | 0.858 |
| ng/ml | (n = 333) | (n = 150) | |
| MCP-1 (urine) | 152.1 ± 199.8 | 216.0 ± 254.1 | <0.001 |
| pg/ml | (n = 334) | (n = 150) | |
| MCP-1 (serum) | 392.8 ± 131.9 | 397.4 ± 145.2 | 0.639 |
| pg/ml | (n = 331) | (n = 150) | |
| Microalbumin | 41.8 ± 65.1 | 71.5 ± 85.1 | <0.001 |
| (urine) mg/l | (n = 334) | (n = 150) | |
| Midkine (urine) | 395.8 ± 582.8 | 891.2 1199.9 | <0.001 |
| pg/ml | (n = 334) | (n = 150) | |
| MMP-9 (urine) | 14.2 ± 34.1 | 19.2 ± 45.1 | 0.986 |
| ng/ml | (n = 334) | (n = 150) | |
| MMP-9/NGAL | 5.0 ± 11.2 | 7.2 ± 12.9 | 0.009 |
| (urine) ng/ml | (n = 334) | (n = 150) | |
| MMP-9/TIMP-1 | 0.8 ± 4.0 | 2.6 ± 7.1 | <0.001 |
| (urine) ng/ml | (n = 334) | (n = 150) | |
| NGAL (urine) | 421.1 506.2 | 479.2 ± 581.7 | 0.521 |
| ng/ml | (n = 334) | (n = 150) | |
| NSE (urine) | 0.6 ± 2.1 | 5.7 ± 17.3 | <0.001 |
| ng/ml | (n = 334) | (n = 150) | |
| Osmolality (urine) | 566.1 ± 222.6 | 604.8 ± 179.2 | 0.071 |
| mOsm/l | (n = 334) | (n = 150) | |
| PAI-1/tPA (serum) | 8.3 ± 6.7 | 12.2 ± 9.4 | <0.001 |
| ng/ml | (n = 334) | (n = 151) | |
| PERK (urine) | 409.4 ± 330.2 | 409.8 ± 361.9 | 0.67 |
| pg/ml | (n = 333) | (n = 147) | |
| Progranulin (urine) | 14.3 ± 10.1 | 18.9 ± 19.6 | 0.001 |
| ng/ml | (n = 334) | (n = 148) | |
| Prolactin (serum) | 263.7 ± 279.1 | 218.1 ± 164.3 | <0.001 |
| mIU/l | (n = 331) | (n = 150) | |
| Protein (urine) | 0.2 ± 0.3 | 0.4 ± 0.6 | <0.001 |
| mg/ml | (n = 334) | (n = 150) | |
| PSA/TPSA (serum) | 3.4 ± 4.5 | 3.0 ± 5.0 | 0.014 |
| ng/ml | (n = 334) | (n = 151) | |
| S100A4 (serum) | 50.4 ± 31.8 | 60.7 ± 44.1 | 0.024 |
| ng/ml | (n = 334) | (n = 151) | |
| sIL-2Ra (serum) | 0.1 ± 0.1 | 0.1 ± 0.0 | 0.045 |
| ng/ml | (n = 334) | (n = 150) | |
| sIL-6R (urine) | 0.6 ± 0.0 | 0.6 ± 0.0 | 0.18 |
| ng/ml | (n = 334) | (n = 150) | |
| TGFβ1 (urine) | 46.6 ± 150.3 | 111.4 ± 274.1 | <0.001 |
| pg/ml | (n = 334) | (n = 150) | |
| Thrombomodulin | 16.5 ± 10.2 | 18.5 ± 10.0 | 0.029 |
| (urine) pg/ml | (n = 334) | (n = 150) | |
| TNFα (urine) | 4.4 ± 2.3 | 5.8 ± 4.2 | 0.003 |
| pg/ml | (n = 334) | (n = 150) | |
| TNFα (serum) | 4.3 ± 2.5 | 4.0 ± 0.8 | 0.767 |
| pg/ml | (n = 331) | (n = 150) | |
| sTNFRI (urine) | 0.9 ± 0.9 | 1.1 ± 0.8 | 0.007 |
| ng/ml | (n = 334) | (n = 150) | |
| sTNRFII (urine) | 1.9 ± 2.3 | 2.1 ± 2.1 | 0.152 |
| ng/ml | (n = 334) | (n = 150) | |
| TPA (urine) | 1.5 ± 3.3 | 3.2 ± 4.9 | <0.001 |
| ng/ml | (n = 334) | (n = 149) | |
| VEGF (urine) | 179.9 ± 239.1 | 361.0 ± 535.8 | <0.001 |
| pg/ml | (n = 334) | (n = 150) | |
| VEGF (serum) | 234.0 ± 145.7 | 290.9 ± 203.4 | 0.006 |
| pg/ml | (n = 331) | (n = 150) | |
| HDL (serum) | 1.1 ± 0.3 | 1.1 ± 0.3 | 0.322 |
| mmol/l | (n = 325) | (n = 145) | |
| LDL (serum) | 2.7 ± 1.0 | 2.6 ± 1.1 | 0.175 |
| mmol/l | (n = 325) | (n = 145) | |
| Triglycerides (serum) | 1.7 ± 1.1 | 1.5 ± 0.7 | 0.049 |
| mmol/l | (n = 325) | (n = 145) | |
| Cholesterol (serum) | 4.6 ± 1.1 | 4.3 ± 1.2 | 0.046 |
| mmol/l | (n = 325) | (n = 145) | |

TABLE 3
Significance of individual biomarkers between female
controls and female bladder cancer patients

| | Female | | |
|---|---|---|---|
| Biomarker | Con | BC | p |
| (matrix) + units | (Status at FD) | (Status at FD) | value |
| 80HdG (urine) | 44.2 ± 34.9 | 59.4 ± 42.9 | 0.009 |
| ng/ml | (n = 139) | (n = 47) | |
| ACR (urine) | 2.8 ± 5.5 | 7.1 ± 8.9 | <0.001 |
| | (n = 139) | (n = 49) | |
| BTA (urine) | 9.1 ± 18.7 | 38.0 ± 39.8 | <0.001 |
| U/ml | (n = 139) | (n = 49) | |
| CD44 (serum) | 149.4 ± 66.0 | 174.6 ± 83.8 | 0.065 |
| ng/ml | (n = 139) | (n = 48) | |
| CEA (serum) | 2.1 ± 1.8 | 2.6 ± 2.0 | 0.047 |
| ng/ml | (n = 139) | (n = 48) | |
| CK-18 (urine) | 14.9 ± 37.9 | 98.9 ± 266.7 | <0.001 |
| ng/ml | (n = 137) | (n = 45) | |
| CK-20 (urine) | 0.2 ± 0.1 | 0.2 ± 0.1 | 0.232 |
| ng/ml | (n = 139) | (n = 47) | |
| Clusterin (urine) | 148.5 ± 196.8 | 440.4 ± 481.2 | <0.001 |
| ng/ml | (n = 139) | (n = 49) | |
| Creatinine (urine) | 63.9 ± 43.5 | 95.7 ± 64.8 | 0.001 |
| μmol/l | (n = 139) | (n = 49) | |
| CRP (urine) | 1.5 ± 0.5 | 1.7 ± 0.7 | 0.095 |
| mg/ml | (n = 139) | (n = 49) | |
| CRP (serum) | 3.2 ± 6.3 | 3.4 ± 5.5 | 0.484 |
| mg/ml | (n = 133) | (n = 46) | |
| CXCL16 (urine) | 0.0 ± 0.0 | 0.1 ± 0.2 | <0.001 |
| ng/ml | (n = 138) | (n = 47) | |
| Cystatin B (urine) | 14.3 ± 21.2 | 26.2 ± 29.4 | 0.001 |
| ng/ml | (n = 139) | (n = 49) | |
| Cystatin C (urine) | 19.3 ± 25.4 | 39.1 ± 42.3 | <0.001 |
| ng/ml | (n = 139) | (n = 48) | |
| Cystatin C (serum) | 0.9 ± 0.4 | 0.9 ± 0.3 | 0.922 |
| ng/ml | (n = 138) | (n = 47) | |
| D-dimer (urine) | 13.4 ± 67.4 | 167.5 ± 415.2 | <0.001 |
| ng/ml | (n = 139) | (n = 49) | |
| EGF (urine) | 7244.6 ± 11591.0 | 17221.9 ± 21223.4 | <0.001 |
| pg/ml | (n = 139) | (n = 49) | |
| EGF (serum) | 18.0 ± 19.3 | 34.4 ± 34.1 | 0.001 |
| pg/ml | (n = 138) | (n = 47) | |
| FABP-A (serum) | 46.9 ± 27.0 | 51.9 ± 36.7 | 0.824 |
| ng/ml | (n = 139) | (n = 48) | |
| FAS (urine) | 15.4 ± 14.3 | 45.5 ± 114.1 | 0.008 |
| pg/ml | (n = 139) | (n = 49) | |
| GRO (CXCL1) | 110.8 ± 107.1 | 128.5 ± 134.3 | 0.105 |
| (serum) pg/ml | (n = 139) | (n = 48) | |
| HAD (serum) | 0.3 ± 0.2 | 0.4 ± 1.4 | 0.002 |
| U/l | (n = 139) | (n = 48) | |
| IFNγ (urine) | 2.0 ± 0.9 | 2.2 ± 1.2 | 0.113 |
| pg/ml | (n = 139) | (n = 49) | |
| IFNγ (serum) | 2.3 ± 2.8 | 2.2 ± 1.3 | 0.389 |
| pg/ml | (n = 138) | (n = 47) | |
| IL-1α (urine) | 6.1 ± 12.9 | 33.0 ± 81.8 | <0.001 |
| pg/ml | (n = 139) | (n = 49) | |
| IL-1α (serum) | 1.0 ± 2.0 | 0.9 ± 0.3 | 0.038 |
| pg/ml | (n = 138) | (n = 47) | |
| IL-1β (urine) | 8.2 ± 26.0 | 33.1 ± 67.7 | <0.001 |
| pg/ml | (n = 139) | (n = 49) | |
| IL-1β (serum) | 1.4 ± 1.0 | 1.3 ± 0.2 | 0.516 |
| pg/ml | (n = 138) | (n = 47) | |
| IL-2 (urine) | 4.5 ± 0.7 | 4.9 ± 3.3 | 0.776 |
| pg/ml | (n = 139) | (n = 49) | |
| IL-2 (serum) | 5.1 ± 3.7 | 4.7 ± 1.1 | 0.812 |
| pg/ml | (n = 138) | (n = 47) | |
| IL-3 (urine) | 8.0 ± 0.7 | 7.9 ± 0.0 | 0.403 |
| pg/ml | (n = 138) | (n = 49) | |
| IL-4 (urine) | 3.4 ± 0.8 | 3.8 ± 1.9 | 0.015 |
| pg/ml | (n = 139) | (n = 49) | |
| IL-4 (serum) | 3.9 ± 3.9 | 3.3 ± 0.2 | 0.674 |
| pg/ml | (n = 138) | (n = 47) | |
| IL-6 (urine) | 6.9 ± 18.6 | 71.9 ± 178.6 | <0.001 |
| pg/ml | (n = 139) | (n = 49) | |
| IL-6 (serum) | 2.7 ± 2.5 | 3.4 ± 3.6 | 0.107 |
| pg/ml | (n = 138) | (n = 47) | |
| IL-7 (urine) | 2.4 ± 1.9 | 4.3 ± 3.2 | <0.001 |
| pg/ml | (n = 138) | (n = 49) | |
| IL-8 (urine) | 208.6 ± 372.0 | 493.8 ± 522.6 | <0.001 |
| pg/ml | (n = 139) | (n = 49) | |
| IL-8 (serum) | 13.3 ± 8.1 | 15.5 ± 9.3 | 0.178 |
| pg/ml | (n = 138) | (n = 47) | |
| IL-10 (urine) | 1.1 ± 0.4 | 1.4 ± 2.0 | 0.078 |
| pg/ml | (n = 139) | (n = 49) | |
| IL-10 (serum) | 1.2 ± 0.7 | 1.9 ± 3.4 | 0.153 |
| pg/ml | (n = 138) | (n = 47) | |
| IL-12p70 (urine) | 3.3 ± 1.4 | 2.7 ± 0.9 | 0.002 |
| pg/ml | (n = 138) | (n = 49) | |
| IL-13 (urine) | 5.6 ± 1.8 | 7.1 ± 2.7 | <0.001 |
| pg/ml | (n = 138) | (n = 49) | |
| IL-18 (serum) | 230.4 ± 456.1 | 94.2 ± 141.3 | 0.171 |
| pg/ml | (n = 138) | (n = 48) | |
| IL-23 (urine) | 0.0 ± 0.0 | 0.0 ± 0.0 | 0.783 |
| pg/ml | (n = 138) | (n = 49) | |
| LASP-1 (serum) | 408.3 ± 307.1 | 407.1 ± 299.7 | 0.869 |
| pg/ml | (n = 139) | (n = 48) | |
| M30 (serum) | 167.7 ± 153.3 | 144.6 ± 85.5 | 0.957 |
| U/l | (n = 139) | (n = 48) | |
| M2PK (serum) | 38.3 ± 106.9 | 31.2 ± 17.6 | 0.57 |
| ng/ml | (n = 139) | (n = 48) | |
| MCP-1 (urine) | 95.5 ± 136.7 | 181.4 ± 163.3 | <0.001 |
| pg/ml | (n = 139) | (n = 49) | |
| MCP-1 (serum) | 375.5 ± 129.4 | 411.6 ± 160.1 | 0.229 |
| pg/ml | (n = 138) | (n = 47) | |
| Microalbumin | 14.0 ± 36.3 | 61.6 ± 80.1 | <0.001 |
| (urine) mg/l | (n = 139) | (n = 49) | |
| Midkine (urine) | 163.1 ± 269.0 | 881.6 ± 1339.9 | <0.001 |
| pg/ml | (n = 139) | (n = 49) | |
| MMP-9 (urine) | 19.4 ± 50.6 | 31.7 ± 74.5 | 0.196 |
| ng/ml | (n = 139) | (n = 49) | |
| MMP-9/NGAL | 3.9 ± 9.0 | 7.5 ± 10.9 | <0.001 |
| (urine) ng/ml | (n = 139) | (n = 49) | |
| MMP-9/TIMP-1 | 0.1 ± 0.3 | 1.1 ± 4.3 | 0.001 |
| (urine) ng/ml | (n = 139) | (n = 49) | |
| NGAL (urine) | 377.7 ± 491.3 | 627.9 ± 619.0 | 0.001 |
| ng/ml | (n = 139) | (n = 49) | |
| NSE (urine) | 0.4 ± 0.4 | 8.8 ± 30.8 | <0.001 |
| ng/ml | (n = 139) | (n = 49) | |
| Osmolality (urine) | 444.7 ± 231.1 | 507.2 ± 199.7 | 0.05 |
| mOsm/l | (n = 139) | (n = 49) | |
| PAI-1/tPA (serum) | 8.0 ± 7.7 | 13.9 ± 10.9 | <0.001 |
| ng/ml | (n = 139) | (n = 48) | |
| pERK (urine) | 354.6 ± 345.1 | 419.5 ± 388.1 | 0.125 |
| pg/ml | (n = 139) | (n = 47) | |
| Progranulin (urine) | 10.7 ± 10.2 | 16.3 ± 13.1 | 0.001 |
| ng/ml | (n = 139) | (n = 48) | |
| Prolactin (serum) | 254.0 ± 148.6 | 260.2 ± 151.5 | 0.54 |
| mIU/l | (n = 137) | (n = 46) | |
| Protein (urine) | 0.1 ± 0.1 | 0.4 ± 0.9 | <0.001 |
| mg/ml | (n = 139) | (n = 49) | |
| PSA/TPSA (serum) | 0.1 ± 0.0 | 0.0 ± 0.0 | 0.247 |
| ng/ml | (n = 139) | (n = 48) | |
| S100A4 (serum) | 63.8 ± 39.7 | 64.8 ± 48.1 | 0.587 |
| ng/ml | (n = 139) | (n = 48) | |
| sIL-2Ra (serum) | 0.1 ± 0.0 | 0.1 ± 0.1 | 0.472 |
| ng/ml | (n = 139) | (n = 49) | |
| sIL-6R (urine) | 0.6 ± 0.1 | 0.6 ± 0.0 | 0.45 |
| ng/ml | (n = 139) | (n = 49) | |
| TGFβ1 (urine) | 21.7 ± 33.3 | 138.1 ± 534.4 | 0.001 |
| pg/ml | (n = 139) | (n = 49) | |
| Thrombomodulin | 11.5 ± 9.0 | 17.5 ± 10.9 | <0.001 |
| (urine) pg/ml | (n = 139) | (n = 49) | |
| TNFα (urine) | 4.2 ± 2.3 | 5.6 ± 6.4 | 0.078 |
| pg/ml | (n = 139) | (n = 49) | |
| TNFα (serum) | 4.5 ± 3.5 | 4.1 ± 1.2 | 0.455 |
| pg/ml | (n = 138) | (n = 47) | |
| sTNFRI (urine) | 0.5 ± 0.6 | 0.8 ± 0.7 | <0.001 |
| ng/ml | (n = 139) | (n = 49) | |
| sTNRFII (urine) | 1.1 ± 1.4 | 1.4 ± 1.7 | 0.101 |
| ng/ml | (n = 139) | (n = 49) | |
| TPA (urine) | 1.7 ± 4.4 | 1.7 ± 3.7 | 0.006 |
| ng/ml | (n = 139) | (n = 48) | |
| VEGF (urine) | 70.0 ± 56.0 | 255.6 ± 492.6 | <0.001 |
| pg/ml | (n = 139) | (n = 49) | |
| VEGF (serum) | 263.0 ± 194.4 | 300.2 ± 203.3 | 0.15 |
| pg/ml | (n = 138) | (n = 47) | |
| HDL (serum) | 1.5 ± 0.4 | 1.5 ± 0.5 | 0.51 |
| mmol/l | (n = 134) | (n = 46) | |
| LDL (serum) | 3.1 ± 1.1 | 2.7 ± 1.0 | 0.049 |
| mmol/l | (n = 134) | (n = 46) | |
| Triglycerides (serum) | 1.5 ± 0.8 | 1.2 ± 0.6 | 0.006 |
| mmol/l | (n = 134) | (n = 46) | |
| Cholesterol (serum) | 5.2 ± 1.3 | 5.0 ± 1.3 | 0.384 |
| mmol/l | (n = 134) | (n = 46) | |

TABLE 4
Biomarker combinations with the highest AUC values for males

| Biomarker combination | AUC | Sensitivity | Specificity |
|---|---|---|---|
| u_NSE + s_PAI-1/tPA + u_Midkine + u_NGAL + | 0.797 | 0.685 | 0.792 |
| u_MMP-9/TIMP-1 + s_LASP-1 + s_Prolactin | | | |
| u_NSE + s_PAI-1/tPA + u_Midkine + u_NGAL + | 0.795 | 0.718 | 0.728 |
| u_MMP-9/TIMP-1 + s_Prolactin | | | |
| u_NSE + s_PAI-1/tPA + u_Midkine + u_NGAL + | 0.792 | 0.669 | 0.801 |
| u_MMP-9/TIMP-1 + s_LASP-1 + u_CXCL16 + | | | |
| s_Prolactin | | | |
| u_NSE + s_PAI-1/tPA + u_Midkine + u_NGAL + | 0.790 | 0.683 | 0.752 |
| u_MMP-9/TIMP-1 + u_CXCL16 + s_Prolactin | | | |
| u_NSE + s_PAI-1/tPA + u_Midkine + u_NGAL + | 0.788 | 0.720 | 0.737 |
| u_MMP-9/TIMP-1 | | | |
| u_NSE + s_PAI-1/tPA + u_Midkine + u_NGAL + | 0.787 | 0.747 | 0.725 |
| u_MMP-9/TIMP-1 + s_LASP-1 | | | |
| u_NSE + s_PAI-1/tPA + u_Midkine + u_NGAL + | 0.783 | 0.719 | 0.740 |
| u_MMP-9/TIMP-1 + u_CXCL16 | | | |
| u_NSE + s_PAI-1/tPA + u_Midkine + u_NGAL + | 0.782 | 0.740 | 0.728 |
| u_MMP-9/TIMP-1 + s_LASP-1 + u_CXCL16 | | | |
| u_NSE + s_PAI-1/tPA + u_Midkine + u_NGAL + | 0.781 | 0.644 | 0.804 |
| s_LASP-1 + s_Prolactin | | | |
| u_NSE + s_PAI-1/tPA + u_Midkine + u_NGAL + | 0.779 | 0.671 | 0.761 |
| s_Prolactin | | | |
| u_NSE + s_PAI-1/tPA + u_NGAL + u_MMP-9/TIMP- | 0.779 | 0.705 | 0.728 |
| 1 + s_LASP-1 + s_Prolactin | | | |
| u_NSE + u_Midkine + u_NGAL + u_MMP-9/TIMP- | 0.778 | 0.691 | 0.752 |
| 1 + s_LASP-1 + s_Prolactin | | | |
| u_NSE + s_PAI-1/tPA + u_NGAL + u_MMP-9/TIMP- | 0.778 | 0.690 | 0.746 |
| 1 + s_LASP-1 + u_CXCL16 + s_Prolactin | | | |
| u_NSE + s_PAI-1/tPA + u_Midkine + u_NGAL + | 0.776 | 0.634 | 0.804 |
| s_LASP-1 + u_CXCL16 + s_Prolactin | | | |
| u_NSE + s_PAI-1/tPA + u_NGAL + u_MMP-9/TIMP- | 0.776 | 0.669 | 0.749 |
| 1 + u_CXCL16 + s_Prolactin | | | |
| u_NSE + s_PAI-1/tPA + u_Midkine + u_NGAL + | 0.774 | 0.662 | 0.758 |
| u_CXCL16 + s_Prolactin | | | |
| u_NSE + u_Midkine + u_NGAL + u_MMP-9/TIMP- | 0.774 | 0.697 | 0.737 |
| 1 + s_LASP1 + u_CXCL16 + s_Prolactin | | | |
| u_NSE + u_Midkine + u_NGAL + u_MMP-9/TIMP- | 0.773 | 0.745 | 0.701 |
| 1 + s_Prolactin | | | |
| u_NSE + u_Midkine + u_NGAL + u_MMP-9/TIMP- | 0.773 | 0.745 | 0.701 |
| 1 + s_Prolactin | | | |
| u_NSE + s_PAI-1/tPA + u_Midkine + u_NGAL | 0.771 | 0.680 | 0.766 |
| u_NSE + u_Midkine + u_NGAL + u_MMP-9/TIMP- | 0.768 | 0.724 | 0.719 |
| 1 + u_CXCL16 + s_Prolactin | | | |
| u_NSE + s_PAI-1/tPA + u_Midkine + u_NGAL + | 0.767 | 0.664 | 0.766 |
| s_LASP-1 + u_CXCL16 | | | |
| u_NSE + s_PAI-1/tPA + u_NGAL + u_MMP-9/TIMP-1 | 0.766 | 0.687 | 0.743 |
| u_NSE + u_NGAL + u_MMP-9/TIMP-1 + s_LASP1 + | 0.765 | 0.683 | 0.737 |
| u_CXCL16 + s_Prolactin | | | |
| u_NSE + s_PAI-1/tPA + u_NGAL + u_MMP-9/TIMP- | 0.765 | 0.692 | 0.737 |
| 1 + s_LASP-1 + u_CXCL16 | | | |
| u_NSE + s_PAI-1/tPA + u_Midkine + u_MMP- | 0.762 | 0.671 | 0.746 |
| 9/TIMP-1 + s_LASP-1 + s_Prolactin | | | |
| u_NSE + s_PAI-1/tPA + u_Midkine + u_MMP- | 0.761 | 0.638 | 0.779 |
| 9/TIMP-1 + s_Prolactin | | | |
| u_NSE + s_PAI-1/tPA + u_Midkine + s_Prolactin | 0.761 | 0.638 | 0.779 |
| s_PAI-1/tPA + u_Midkine + u_NGAL + u_MMP- | 0.761 | 0.678 | 0.722 |
| 9/TIMP-1 + s_LASP-1 + s_Prolactin | | | |
| u_NSE + u_Midkine + u_NGAL + u_MMP-9/TIMP-1 | 0.761 | 0.647 | 0.751 |
| u_NSE + u_Midkine + u_NGAL + u_MMP-9/TIMP- | 0.760 | 0.603 | 0.823 |
| 1 + s_LASP-1 + u_CXCL16 | | | |
| s_PAI-1/tPA + u_Midkine+ u_NGAL + u_MMP- | 0.760 | 0.705 | 0.695 |
| 9/TIMP-1 + s_Prolactin | | | |
| u_NSE + s_PAI-1/tPA + u_NGAL + s_Prolactin | 0.759 | 0.671 | 0.737 |
| u_NSE + s_PAI-1/tPA + u_NGAL + s_LASP-1 + | 0.758 | 0.641 | 0.758 |
| u_CXCL16 + s_Prolactin | | | |
| u_NSE + u_NGAL + u_MMP-9/TIMP-1 + s_Prolactin | 0.757 | 0.718 | 0.674 |
| u_NSE + u_NGAL + u_MMP-9/TIMP-1 + s_Prolactin | 0.757 | 0.718 | 0.674 |
| s_PAI-1/tPA + u_Midkine + u_NGAL + u_MMP- | 0.757 | 0.662 | 0.728 |
| 9/TIMP-1 + s_LASP-1 + u_CXCL16 + s_Prolactin | | | |
| s_PAI-1/tPA + u_Midkine + u_NGAL + u_MMP- | 0.755 | 0.690 | 0.710 |
| 9/TIMP-1 + u_CXCL16 + s_Prolactin | | | |
| u_NSE + s_PAI-1/tPA + u_Midkine + u_MMP- | 0.755 | 0.655 | 0.740 |
| 9/TIMP-1 + u_CXCL16 + s_Prolactin | | | |
| u_NSE + s_PAI-1/tPA + u_Midkine + u_MMP- | 0.755 | 0.662 | 0.746 |
| 9/TIMP-1 + s_LASP-1 + u_CXCL16 + s_Prolactin | | | |
| u_NSE + s_PAI-1/tPA + u_Midkine + s_LASP-1 + | 0.755 | 0.662 | 0.746 |
| u_CXCL16 + s_Prolactin | | | |
| s_PAI-1/tPA + u_Midkine + u_NGAL + u_MMP- | 0.754 | 0.693 | 0.683 |
| 9/TIMP-1 | | | |
| u_NSE + s_PAI-1/tPA + s_Prolactin | 0.752 | 0.705 | 0.698 |
| u_NSE + s_PAI-1/tPA + u_MMP-9/TIMP-1 + | 0.752 | 0.664 | 0.743 |
| s_Prolactin | | | |
| u_NSE + s_PAI-1/tPA + u_Midkine + u_MMP- | 0.750 | 0.640 | 0.754 |
| 9/TIMP-1 | | | |
| s_PAI-1/tPA + u_Midkine + u_NGAL + u_MMP- | 0.750 | 0.699 | 0.671 |
| 9/TIMP-1 + s_LASP-1 + u_CXCL16 | | | |
| u_NSE + s_PAI-1/tPA + u_Midkine | 0.750 | 0.640 | 0.754 |
| u_NSE + s_PAI-1/tPA + u_MMP-9/TIMP-1 + | 0.749 | 0.628 | 0.776 |
| s_LASP-1 + u_CXCL16 + s_Prolactin | | | |
| u_NSE + u_Midkine + u_NGAL + s_LASP-1 + | 0.748 | 0.731 | 0.622 |
| u_CXCL16 + s_Prolactin | | | |
| u_NSE + u_Midkine + u_NGAL + s_Prolactin | 0.746 | 0.624 | 0.752 |
| u_NSE+ s_PAI-1/tPA + u_NGAL | 0.745 | 0.667 | 0.754 |
| u_NSE + s_PAI-1/tPA + u_Midkine + u_MMP- | 0.742 | 0.726 | 0.659 |
| 9/TIMP-1 + s_LASP-1 + u_CXCL16 | | | |
| s_PAI-1/tPA + u_Midkine + u_NGAL + s_Prolactin | 0.739 | 0.638 | 0.752 |
| s_PAI-1/tPA + u_Midkine + u_NGAL + s_LASP-1 + | 0.735 | 0.634 | 0.734 |
| u_CXCL16 + s_Prolactin | | | |
| s_PAI-1/tPA + u_NGAL + u_MMP-9/TIMP-1 + | 0.735 | 0.614 | 0.728 |
| s_LASP-1 + u_CXCL16 + s_Prolactin | | | |
| u_NSE + s_PAI-1/tPA | 0.733 | 0.647 | 0.757 |
| s_PAI-1/tPA + u_Midkine + u_NGAL | 0.730 | 0.667 | 0.707 |
| u_Midkine + u_NGAL + u_MMP-9/TIMP-1 + | 0.729 | 0.752 | 0.601 |
| s_LASP-1 + u_CXCL16 + s_Prolactin | | | |
| s_PAI-1/tPA + u_Midkine + s_Prolactin | 0.728 | 0.658 | 0.716 |
| s_PAI-1/tPA + u_Midkine + u_MMP-9/TIMP-1 + | 0.728 | 0.644 | 0.716 |
| s_Prolactin | | | |
| u_NSE + u_Midkine + u_MMP-9/TIMP-1 + | 0.728 | 0.607 | 0.713 |
| s_LASP-1 + u_CXCL16 + s_Prolactin | | | |
| u_NSE + u_NGAL + s_Prolactin | 0.726 | 0.691 | 0.647 |
| u_NSE + u_Midkine + s_Prolactin | 0.726 | 0.617 | 0.713 |
| u_Midkine + u_NGAL + u_MMP-9/TIMP-1 + | 0.726 | 0.638 | 0.698 |
| s_Prolactin | | | |
| u_NSE + u_Midkine + u_NGAL | 0.725 | 0.613 | 0.722 |
| s_PAI-1/tPA + u_Midkine + u_MMP-9/TIMP-1 + | 0.723 | 0.641 | 0.695 |
| s_LASP-1 + u_CXCL16 + s_Prolactin | | | |
| s_PAI-1/tPA + u_NGAL + u_MMP-9/TIMP-1 + | 0.720 | 0.664 | 0.686 |
| s_Prolactin | | | |
| u_NSE + s_Prolactin | 0.719 | 0.738 | 0.583 |
| s_PAI-1/tPA + u_Midkine | 0.716 | 0.660 | 0.689 |
| u_NSE + u_Midkine | 0.702 | 0.607 | 0.674 |
| u_NSE + s_LASP-1 | 0.698 | 0.673 | 0.638 |

*‘u’ and ‘s’ indicate urine and serum biomarkers

### DESCRIPTION

The present invention is based on the finding that levels of certain biomarkers present in a male patient suffering from bladder cancer, enable a more accurate diagnosis to be made compared to the prior methods of diagnosis based on biomarkers that are used for the diagnosis of both men and women. Identification of particular biomarkers in a sample isolated from a male patient is indicative of the susceptibility to or the presence of cancer in the male patient, and it has been surprisingly found that these biomarkers differ significantly in men and women.

As used herein, the term ‘biomarker’ refers to a molecule present in a biological sample obtained from a patient, the concentration of which in said sample may be indicative of a pathological state. Various biomarkers that have been found to be useful in diagnosing bladder cancer, either alone or in combination with other diagnostic methods, or as complementary biomarkers in combination with other biomarkers, are described herein. Diagnosis may be made based on the level of expression or the concentration of the biomarker in a sample isolated from the patient. The biomarkers of the present invention are typically identified in a serum or urine sample isolated from the patient. The preferred sample type can be dependent on the biomarker being measured. For example, in the current invention the biomarkers NSE, Midkine, NGAL, MMP-9/TIMP-1 and CXCL16 are preferably measured in a urine sample, while the biomarkers PAI-1/tPA, LASP-1 and Prolactin are preferably measured in a serum sample.

In one embodiment the current invention provides a method for the detection of, or determining the risk of, bladder cancer in a male patient, the method comprising determining the level of a panel of biomarkers in a sample previously isolated from a male patient. The phrase “determining the level of a panel of biomarkers” as used herein, means determining the concentration of two or more biomarkers in a patient sample. Said two or more biomarkers make up the panels of the invention. The panel of biomarkers with which the present invention is concerned comprises one or both of prolactin and LASP-1 and one or more additional biomarkers selected from NSE, PAI-1/tPA, MMP-9/TIMP-1, NGAL, Midkine, ACR, BTA, CD44, CEA, CK-18, CK-20, Clusterin, Creatinine, CRP, CXCL16, Cystatin B, Cystatin C, D-dimer, EGF, FABP-A, FAS, CXCL1, IFNγ, IL-1α, IL-6, IL-7, IL-8, MCP-1, Microalbumin, MMP-9/NGAL, osmolality, PAI-1/tPA, Progranulin, TUP, PSA_TPSA, S100A4, TGFβ1, sTNFR1, sTNFR2, thrombomodulin, TNFα, tPA, VEGF and cholesterol. Preferably the one or more additional biomarkers are selected from NSE, PAI-1/tPA, Midkine, NGAL, MMP-9/TIMP-1, and CXCL16. Preferred combinations of the current invention include prolactin and NSE and at least one additional biomarker selected from PAI-1/tPA, NGAL, MMP-9/TIMP-1, and Midkine. Even more preferably the panel of biomarkers comprises one of the following combinations:
i) prolactin and NSE
ii) prolactin, NSE and PAI-1/tPA
iii) prolactin, NSE, NGAL and MMP-9/TIMP-1
iv) prolactin, NSE, PAI-1/tPA and NGAL
v) prolactin, NSE, PAI-1/tPA and midkine
vi) prolactin, NSE, PAI-1/tPA, midkine and NGAL
vii) prolactin, NSE, PAI-1/tPA, midkine, NGAL, and MMP-9/TIMP-1.

These combinations and other preferred combinations of the current invention are shown in Table 4.

In this embodiment the method further comprises assessing the presence or risk of bladder cancer in the male patient wherein detection of an altered level of the biomarkers compared to a normal control indicates the presence or the risk of cancer in the male patient from whom the sample has previously been isolated.

Taking gender into account has been demonstrated herein as an important factor for single biomarkers as well as for models comprising multiple biomarkers. For example, several serum biomarkers were found to only be significantly different between controls and bladder cancer patients in males. These were IL-10, PSA_TPSA and S100A4 (Table 2). Several urine biomarkers, CK-20, IFNγ, and TNFα, were significantly different overall (without gender separation) and in males but not in females (Tables 1-3). Several serum biomarkers, CRP, CXCL1, LASP-1, prolactin, IL-2Ra, VEGF and cholesterol, were also only significantly different overall and in males (Tables 1-3). While expression of individual biomarkers may be different between genders, biomarkers may also contribute differently to biomarker models in each gender. For example, a biomarker which significantly contributes to a model in males may not significantly contribute to the same model in females.

Therefore, one embodiment of the current invention is the use of those biomarkers from Tables 1 to 3 (IL-10, PSA_TPSA, S100A4, CK-20, IFNγ, TNFα, CRP, CXCL1, LASP-1, prolactin, IL-2Ra, VEGF and cholesterol) which were found to only be significantly different between bladder cancer and controls in males or overall (without gender separation), in the diagnosis of bladder cancer in males. Preferably this includes their use in multi-biomarker models.

In some embodiments the sample has a concentration of albumin and creatinine expressed as an albumin: creatinine ratio (ACR). This may be calculated by measuring the concentration separately of albumin and creatinine. The skilled person will appreciate conventional ways to measure albumin and creatinine concentrations, see examples for illustrative methods. When the kidneys are functioning properly there is virtually no albumin present in the urine.

In some embodiments the patient may be presenting with haematuria and/or with an infection. For the avoidance of doubt, the term ‘haematuria’ refers to the presence of red blood cells in the urine. Suitably the infection may be a bacterial or viral infection, preferably a bacterial infection. Suitably, the method may further comprise a step of characterising the patient's infection status. Characterising infection means diagnosing the patient as having infection or being infection free and may include identifying the infecting species. Infection may be determined using clinical-based diagnoses based on clinical history, biomarkers, dipstick analysis or UTI multiplex array (e.g., Randox Urinary Track Multiplex Assay). By incorporating an initial infection test the AUCs can be increased and also potentially reduce the number of biomarkers used to diagnose the bladder cancer.

In the context of the present invention, the term ‘bladder cancer’ is understood to include urothelial carcinoma (UC), transitional cell papillary carcinoma, transitional cell carcinoma, bladder squamous cell carcinoma, bladder adenocarcinoma and/or bladder sarcoma. In some embodiments the presence of haematuria and/or an infection may further increase the elevated levels of the biomarkers within the panel of biomarkers compared to if haematuria and/or the infection were not present in male bladder cancer patients.

In a preferred embodiment, the biomarkers within the panel may be identified and their concentrations within the sample determined either sequentially or simultaneously in the sample isolated from the patient. The biomarkers may be identified and their concentrations within the isolated sample may be determined by routine methods, which are known in the art, such as by contacting the sample with a substrate having binding molecules specific for each of the biomarkers included in the panel of biomarkers. Preferably the substrate has at least two binding molecules immobilised thereon, more preferably three, four or more binding molecules, wherein each binding molecule is specific to an individual biomarker and the first probe is specific for prolactin and the second probe is specific to NSE.

As used herein, the term ‘specific’ means that the binding molecule binds only to one of the biomarkers of the invention, with negligible binding to other biomarkers of the invention or to other analytes in the biological sample being analysed. This ensures that the integrity of the diagnostic assay and its result using the biomarkers of the invention is not compromised by additional binding events.

The biomarker concentrations may be measured by using methodology based on immuno-detection. As such, the binding molecule is preferably an antibody, such as a polyclonal antibody or a monoclonal antibody. As used herein, the term ‘antibody’ includes any immunoglobulin or immunoglobulin-like molecule or fragment thereof, Fab fragments, ScFv fragments and other antigen binding fragments. The term ‘polyclonal antibodies’ refers to a heterogeneous population of antibodies which recognise multiple epitopes on a target/antigen. The term ‘monoclonal antibodies’ refers to a homogenous population of antibodies (including antibody fragments), which recognise a single epitope on a target/antigen.

Immuno-detection technology is also readily incorporated into transportable or hand-held devices for use outside of the clinical environment. A quantitative immunoassay such as a Western blot or ELISA can be used to detect the amount of protein biomarkers. A preferred method of analysis comprises using a multianalyte biochip which enables several proteins to be detected and quantified simultaneously. 2D Gel Electrophoresis is also a technique that can be used for multi-analyte analysis.

In a preferred embodiment, the binding molecules are immobilised on a solid support, ready to be contacted with the patient sample. A preferred solid support material is in the form of a biochip. A biochip is typically a planar substrate that may be, for example, mineral or polymer based, but is preferably ceramic. The solid support may be manufactured according to the method disclosed in, for example, GB-A-2324866 the contents of which is incorporated herein in its entirety. The solid supports may be screen printed in accordance with known methods disclosed in, for example, WO2017/085509. Preferably, the Biochip Array Technology system (BAT) (available from Randox Laboratories) may be used to determine the levels of biomarkers in the sample. More preferably, the Evidence Evolution and Evidence Investigator apparatus (available from Randox Laboratories) may be used.

The solid support material comprises binding molecules attached thereto, said binding molecules having affinity specific for one or both of prolactin and, separately, LASP-1, with the binding molecules each being in discrete locations on the support material. The solid support material may further comprise, each in discrete locations, one or more binding molecules each having affinity specific for an additional biomarker selected from ACR, BTA, CD44, Midkine, PAI-1/tPA, CEA, CK-18, CK-20, Clusterin, Creatinine, CRP, CXCL16, Cystatin B, Cystatin C, D-dimer, EGF, FABP-A, FAS, CXCL1, IFNγ, IL-1α, IL-6, IL-7, IL-8, MCP-1, Microalbumin, Midkine, MMP-9/NGAL, MMP-9/TIMP-1, NSE, osmolality, PAI-1/tPA, Progranulin, TUP, free PSA, total PSA, S100A4, TGFβ1, sTNFR1, STNFR2, thrombomodulin, TNFα, VEGF and cholesterol. Preferably the one or more additional biomarkers are selected from NSE, PAI-1/tPA, Midkine, NGAL, MMP-9/TIMP-1, and CXCL16. Even more preferably the binding molecules attached to the solid support have affinities to NSE, PAI-1/tPA, Midkine, NGAL, MMP-9/TIMP-1, and Prolactin.

The present invention also provides the use of the substrate described in a method for the detection of or the risk of bladder cancer in a male patient.

The present invention also provides kits comprising probes for a panel of biomarkers comprising one or both of prolactin and LASP-1 and one or more biomarkers selected from NSE, PAI-1/tPA, NGAL, MMP-9/TIMP-1, Midkine, ACR, BTA, CD44, CEA, CK-18, CK-20, Clusterin, Creatinine, CRP, CXCL16, Cystatin B, Cystatin C, D-dimer, EGF, FABP-A, FAS, CXCL1, IFNγ, IL-1α, IL-6, IL-7, IL-8, MCP-1, Microalbumin, MMP-9/NGAL, osmolality, Progranulin, TUP, free PSA, total PSA, S100A4, TGFβ1, STNFR1, STNFR2, thrombomodulin, TNFα, VEGF and cholesterol. Preferably prolactin and one or more of NSE, PAI-1/tPA, Midkine, NGAL, and MMP-9/TIMP-1. Such kits can be used to detect bladder cancer or the risk of bladder cancer in a male patient according to the first aspect of the invention.

Prolactin as used herein refers to the UniProt number P01236, NSE as used herein refers to UniProt number P09104, PAI-1/tPA as used herein refers to the complex between UniProt numbers P05121 and P00750, Midkine as used herein refers to UniProt number P21741, NGAL as used herein refers to UniProt number P80188, MMP9TIMP1 as used herein refers to the complex between UniProt numbers P14780 and P01033, PSA_tPSA as used herein refers to the ratio between free and total PSA (UniProt number P07288), IL-10 as used herein refers to UniProt number P22301, S100A4 as used herein refers to UniProt number 26447, LASP-1 as used herein refers to UniProt number Q14847 and CXCL16 as used herein refers to UniProt number Q9H2A7.

The invention also provides a method for the detection of or the risk of bladder cancer in a male patient comprising the steps of
(i) determining that a male patient does not have an infection;
(ii) detecting the presence of prolactin and one or more additional biomarkers in a sample isolated from the male patient, wherein said one or more additional biomarkers are selected from LASP-1, NSE, PAI-1/tPA, Midkine, NGAL, and MMP-9/TIMP-1;
(iii) assessing the presence or risk of bladder cancer in the male patient wherein detection of an elevated presence of the biomarkers compared to a normal control indicates the presence or the risk of cancer in the male patient from whom the sample is isolated.

Suitably, the one or more biomarkers are prolactin, NSE, PAI-1/tPA, Midkine, NGAL, and MMP-9/TIMP-1.

In the methods of the present invention, in order for bladder cancer or the risk of bladder cancer to be diagnosed, the biomarkers within the panel of biomarkers tested may be found at an elevated level compared to the corresponding biomarker in a normal control sample. In some embodiments, the concentrations of biomarkers are found at a significantly higher level than in a control sample. The determination of “higher concentration” is relative and determined with respect to a control subject known not to have bladder cancer. Biomarkers found herein to have higher concentrations in male bladder cancer patients than in controls included NSE, PAI-1/tPA, Midkine, NGAL, MMP-9/TIMP-1, S100A4, LASP-1 and CXCL16. For some biomarkers the concentrations are found at a significantly lower level than in a control sample. The determination of “lower concentration” is relative and determined with respect to a control subject known not to have bladder cancer. Biomarkers found herein to have lower concentrations in male bladder cancer patients than in controls included prolactin, PSA_tPSA, and IL-10.

Control values are derived from the concentration of corresponding biomarkers in a biological sample obtained from an individual or individuals who do not have bladder cancer. Such individual(s) may be, for example, healthy individuals or individuals suffering from diseases other than bladder cancer. Alternatively, the control values may correspond to the concentration of each of the biomarkers in a sample obtained from the patient prior to getting bladder cancer. For the avoidance of doubt, the term ‘corresponding biomarkers’ means that concentrations of the same biomarker or combination of biomarkers that are determined in respect of the patient's sample are also used to determine the control values. For example, if the concentration of prolactin and NSE is determined in the patient's sample, then the concentration of prolactin and NSE in the control is also known.

A biomarker present in a sample isolated from a male patient having bladder cancer may have levels which are different to that of a control. However, the levels of some biomarkers that are different compared to a control may not show a strong enough correlation with bladder cancer such that they may be used to diagnose cancer with an acceptable accuracy. When two or more biomarkers are to be used in the diagnostic method a suitable mathematical or machine learning classification model, such as logistic regression equation, can be derived. Such models as described herein may be referred to as “statistical methodologies”. The significance of the levels of the biomarkers can be established by inputting into said model. Such a classification model may be chosen from at least one of decision trees, artificial neural networks, logistic regression, random forests, support vector machine or indeed any other method developing classification models known in the art. The output of the models used herein would correlate with the risk of a male patient having or developing bladder cancer. Such an output could be a numerical value, for example a number between 0 and 1, an odds ratio value, a risk ratio/relative risk value or an alphabetic output such as ‘yes’ or ‘no’ or ‘high risk’, ‘low risk’ etc.

Variables can be logarithmically, or square root transformed in a regression model when data is not normally distributed. Table 5 below shows some suitable biomarker models of the invention.

TABLE 5
Biomarker models derived for diagnosis of bladder cancer in male patients

| Model Formula | AUC | Sensitivity | Specificity |
|---|---|---|---|
| Status.at.FD = 2.243 + 0.681*u_NSE_log + | 0.795 | 0.718 | 0.728 |
| 0.491*s_PAI-1/tPA_sqrt + 0.041*u_Midkine_sqrt + | | | |
| −0.002*u_NGAL + 0.38*u_MMP-9/TIMP-1_log + | | | |
| −0.607*s_Prolactin_log | | | |
| Status.at.FD = 1.149 + 0.701*u_NSE_log + | 0.779 | 0.671 | 0.761 |
| 0.51*s_PAI-1/tPA_sqrt + 0.044*u_Midkine_sqrt + | | | |
| −0.001*u_NGAL + −0.643*s_Prolactin_log | | | |
| Status.at.FD = 0.992 + 0.599*u_NSE_log + | 0.761 | 0.638 | 0.779 |
| 0.479*s_PAI-1/tPA_sqrt + 0.027*u_Midkine_sqrt + | | | |
| −0.626*s_Prolactin_log | | | |
| Status.at.FD = 1.856 + 0.836*u_NSE_log + | 0.759 | 0.671 | 0.737 |
| 0.461*s_PAI-1/tPA_sqrt + −0.001*u_NGAL + | | | |
| −0.602*s_Prolactin_log | | | |
| Status.at.FD = 4.839 + 0.791*u_NSE_log + | 0.757 | 0.718 | 0.674 |
| −0.002*u_NGAL + 0.448*u_MMP-9/TIMP-1_log + | | | |
| −0.65*s_Prolactin_log | | | |
| Status.at.FD = 1.582 + 0.734*u_NSE_log + | 0.752 | 0.705 | 0.698 |
| 0.452*s_PAI-1/tPA_sqrt + −0.603*s_Prolactin_log | | | |
| Status.at.FD = 3.369 + 0.751*u_NSE_log + | 0.719 | 0.738 | 0.583 |
| −0.682*s_Prolactin_log | | | |

FD = final diagnosis for patient dataset

A Clinical Risk Score (CRS) can be calculated for the patient, which is a cumulative score using, but not restricted to, the following clinical and demographic measurements: age, haematuria (non-visible vs. macro haematuria), smoking (pack years), BMI, blood pressure (controlled, normotensive, hypertensive), occupational risk score (FINJEM), social class (ONS Codes), comorbidities e.g. diabetes, chronic kidney disease (CKD) etc., medications e.g. statins, anti-hypertensives etc, specific medications (found to increase risk of bladder cancer), pain relief, renal transplant, kidney cancer, other cancers, pelvic radiotherapy and UTIs (with/without microbiology). Example scores used when calculating the CRS for a patient: age is greater than 65 equals a score of 1; age is less than 65 equals a score of 0; non-visible haematuria (NVH) equals a score of 1; macro haematuria equals a score of 2. Therefore, a patient who is older than 65 years with macro haematuria would have a cumulative score of 3, using age and haematuria as clinical risk scores. The biochip bladder cancer test data and CRS is combined to determine if the patient was in one of the following categories: low risk, medium risk, or high risk. This information would allow the GP to manage their patients in primary care and refer them for further tests if and when appropriate. For example, patients who present with haematuria and are negative by biochip and have a low CRS could be monitored in primary care by their GP, rather than being referred to have a cystoscopy. Patients who are negative by biochip and have a moderate CRS could be referred to urology for cystoscopy (non-urgent). Patients who are positive by biochip and have a low CRS could be referred to urology for cystoscopy (non-urgent). Patients who are positive by biochip and have moderate CRS could be ‘red flagged’ for an urgent cystoscopy (Table 6).

TABLE 6
Biomarker Risk Score and Clinical Risk Score

| | Biomarker risk score (BRS) | | Clinical risk score (CRS) | |
|---|---|---|---|---|
| | Negative | Positive | Low | Moderate |
| Monitor at 3, 6, | Yes | No | Yes | No |
| 9 & 12 months | | | | |
| using BRS | | | | |
| and CRS | | | | |
| Referral for | Yes | No | No | Yes |
| Cystoscopy | | | | |
| (non-urgent) | | | | |
| Referral for | No | Yes | Yes | No |
| Cystoscopy | | | | |
| (non-urgent) | | | | |
| Urgent referral | No | Yes | No | Yes |
| for Cystoscopy | | | | |

When used in combination with CRS the BRS improved the negative predictive value significantly (Table 7), this would be an important contribution to the triage of patients potentially allowing bladder cancer to be ruled out as a diagnosis without the need for cystoscopy.

TABLE 7
Performance of clinical risk score (CRS) and biomarker
risk score (BRS) individually and in combination

| | % PPV (95% CL) | % NPV (95% CL) |
|---|---|---|
| CRS | 36.5 (31.9-41.3) | 81.4 (76.1-86.0) |
| Male BRS | 52.6 (45.9-59.2) | 88.8 (84.2-92.4) |
| Male BRS and CRS | 37.7 (32.9-42.7) | 97.8 (92.2-99.7) |

The accuracy of statistical methods used in accordance with the present invention can be best described by their receiver operating characteristics (ROC). The ROC curve addresses both the sensitivity, the number of true positives, and the specificity, the number of true negatives, of the test. Therefore, sensitivity and specificity values for a given combination of biomarkers are an indication of the accuracy of the assay. For example, if a biomarker combination has sensitivity and specificity values of 80%, out of 100 patients which have bladder cancer, 80 will be correctly identified from the determination of the presence of the particular combination of biomarkers as positive for bladder cancer, while out of 100 patients who have not got bladder cancer 80 will accurately test negative for the disease. The ROC also provides a measure of the predictive power of the test in the form of the area under the curve (AUC). AUC is a measure of the probability that the perceived measurement will allow correct identification of a condition. By convention, this area is always ≥0.5. Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0). In a preferred embodiment, the panel of biomarkers has an AUC value of at least 0.7.

It is well understood in the art that biomarker normal or ‘background’ concentrations may exhibit slight variation due to, for example, age, gender, or ethnic/geographical genotypes. As a result, the cut-off value used in the methods of the invention may also slightly vary due to optimization depending upon the target patient or population. Adjusting the cut-off will also allow the operator to increase the sensitivity at the expense of specificity and vice versa. In one embodiment, the algorithm has a sensitivity and/or specificity of at least 0.7 respectively.

Where two or more biomarkers are used in the invention, a suitable mathematical or machine learning classification model, such as logistic regression equation, can be derived. The skilled statistician will understand how such a suitable model is derived, which can include other variables such as age and gender of the patient. The ROC curve can be used to assess the accuracy of the model, and the model can be used independently or in an algorithm to aid clinical decision making. Although a logistic regression equation is a common mathematical/statistical procedure used in such cases and an option in the context of the present invention, other mathematical/statistical, decision trees or machine learning procedures can also be used. The skilled person will appreciate that the model generated for a given population may need to be adjusted before application to datasets obtained from different populations or patient cohorts.

1. A method for the detection of, or determining the risk of, bladder cancer in a male patient comprising the steps of:
(i) determining the level of a panel of biomarkers in a sample previously isolated from a male patient, said panel of biomarkers comprising one or both of prolactin and LIM And SH3 Protein 1 (LASP-1) and one or more additional biomarker selected from neuron specific enolase (NSE), Plasminogen Activator Inhibitor 1/Tissue Plasminogen Activator complex (PAI-1/tPA), Matrix Metalloprotein 9/Tissue Inhibitor of Metalloprotein 1 complex (MMP-9/TIMP-1), Neutrophil Gelatinase Associated Lipocalin Complex (NGAL), Midkine, Albumin: creatinine ratio (ACR), bladder tumour antigen (BTA), cluster of differentiation 44 (CD44), carcinoembryonic antigen (CEA), cytokeratin 18 (CK-18), cytokeratin 20 (CK-20), Clusterin, Creatinine, C reactive protein (CRP), C-X-C motif chemokine ligand 1 (CXCL1), C-X-C Motif Chemokine Ligand 16 (CXCL16), Cystatin B, Cystatin C, D-dimer, epidermal growth factor (EGF), fatty acid-binding protein A (FABP-A), FAS protein (FAS), interferon gamma (IFNγ), interleukin 1α (IL-1α), interleukin 2 receptor alpha chain (IL-2Ra), interleukin 6 (IL-6), interleukin 7 (IL-7), interleukin 8 (IL-8), interleukin-10 (IL-10), monocyte chemotactic protein 1 (MCP-1), Microalbumin, Matrix Metalloprotein-9/Neutrophil Gelatinase-Associated Lipocalin Complex (MMP-9/NGAL), Osmolality, Progranulin, total urinary Protein, free prostate-specific antigen to total prostate specific antigen ratio (PSA_TPSA), Protein S100-A4 (S100A4), Transforming growth factor beta 1 (TGFβ1), Tumour necrosis factor alpha (TNFα), soluble tumour necrosis factor receptor 1 (sTNFR1), soluble tumour necrosis factor receptor 2 (STNFR2), Tissue Type Plasminogen Activator (TPA), Vascular Endothelial Growth Factor (VEGF) and cholesterol;
(ii) assessing the presence or risk of bladder cancer in the male patient wherein detection of an altered level of the biomarkers compared to a normal control indicates the presence or the risk of cancer in the male patient from whom the sample is isolated.

2. The method of claim 1 wherein the one or more additional biomarkers are selected from NSE, PAI-1/tPA, Midkine, NGAL, CXCL16 and MMP-9/TIMP-1.

3. The method of claim 1, wherein the panel of biomarkers comprises
i) prolactin and NSE
ii) prolactin, NSE and PAI-1/tPA
iii) prolactin, NSE, NGAL and MMP-9/TIMP-1
iv) prolactin, NSE, PAI-1/tPA and NGAL
v) prolactin, NSE, PAI-1/tPA and Midkine
vi) prolactin, NSE, PAI-1/tPA, Midkine and NGAL
vii) prolactin, NSE, PAI-1/tPA, Midkine, NGAL, and MMP-9/TIMP-1.

4. The method of claim 1, wherein one or more of the biomarkers are measured in a urine sample and one or more of the biomarkers are measured in a serum sample.

5. The method of claim 3 wherein the biomarkers NSE, Midkine, NGAL, and MMP-9/TIMP-1 are measured in a urine sample.

6. The method of claim 3 wherein the biomarkers PAI-1/tPA and Prolactin are measured in a serum sample.

7. The method of claim 1, wherein the method further comprises a step of characterising the patient's infection status.

8. The method of claim 1, wherein step (ii) comprises inputting the measured concentrations of the biomarkers from step (i) into an algorithm such that the output of the algorithm indicates whether the individual has or is at risk of developing bladder cancer, preferably wherein the output of the algorithm has a sensitivity of at least 70% and/or wherein the output of the algorithm has a specificity of at least 70%.

9. The method of claim 1, wherein the patient presented with haematuria.

10. A solid support material comprising binding molecules attached thereto, said binding molecules having affinity specific for prolactin with the binding molecules being in discrete locations on the support material.

11. The solid support material according to claim 10, further comprising, each in discrete locations, binding molecules for one or more biomarker selected from neuron specific enolase (NSE), Plasminogen Activator Inhibitor 1/Tissue Plasminogen Activator complex (PAI-1/tPA), Matrix Metalloprotein 9/Tissue Inhibitor of Metalloprotein 1 complex (MMP-9/TIMP-1), Neutrophil Gelatinase Associated Lipocalin Complex (NGAL), Midkine, Albumin: creatinine ratio (ACR), bladder tumour antigen (BTA), cluster of differentiation 44 (CD44), carcinoembryonic antigen (CEA), cytokeratin 18 (CK-18), cytokeratin 20 (CK-20), Clusterin, Creatinine, C reactive protein (CRP), C-X-C motif chemokine ligand 1 (CXCL1), C-X-C Motif Chemokine Ligand 16 (CXCL16), Cystatin B, Cystatin C, D-dimer, epidermal growth factor (EGF), fatty acid-binding protein A (FABP-A), FAS protein (FAS), interferon gamma (IFNγ), interleukin 1α (IL-1α), interleukin 2 receptor alpha chain (IL-2Ra), interleukin 6 (IL-6), interleukin 7 (IL-7), interleukin 8 (IL-8), interleukin-10 (IL-10), monocyte chemotactic protein 1 (MCP-1), Microalbumin, Matrix Metalloprotein-9/Neutrophil Gelatinase-Associated Lipocalin Complex (MMP-9/NGAL), Osmolality, Progranulin, total urinary Protein, free prostate-specific antigen to total prostate specific antigen ratio (PSA TPSA), Protein S100-A4 (S100A4), Transforming growth factor beta 1 (TGFβ1), Tumour necrosis factor alpha (TNFα), soluble tumour necrosis factor receptor 1 (sTNFR1), soluble tumour necrosis factor receptor 2 (sTNFR2), Tissue Type Plasminogen Activator (TPA), Vascular Endothelial Growth Factor (VEGF) and cholesterol.

12. The solid support material according to claim 11, wherein the binding molecules, separately, have affinity for the biomarkers NSE, PAI-1/tPA, Midkine, NGAL, MMP-9/TIMP-1, and Prolactin.

13. The solid support material according to claim 10, wherein the binding molecules are antibodies.

14. The solid support material according to claim 10, wherein the support is a biochip.

15. A method for the detection of, or determining the risk of, bladder cancer in a male patient comprising the steps of:
(i) determining that a male patient does not have an infection;
(ii) detecting the presence of a panel of biomarkers in a sample previously isolated from the male patient, said panel of biomarkers comprising Prolactin or LASP-1, and one or more biomarkers selected from NSE, PAI-1/tPA, Midkine, NGAL, CXCL16 and MMP-9/TIMP-1;
(iii) assessing the presence or risk of bladder cancer in the male patient wherein detection of an altered level of the biomarkers compared to a normal control indicates the presence or the risk of cancer in the male patient from whom the sample is isolated.

16. The solid support material of claim 10, further comprising a binding molecule to LASP-1, wherein the binding molecules are at discrete locations on the support material.
